(19) United States
(12) Patent Application Publication
(10) Pub. No.: US 2010/0113557 A1
(43) Pub. Date: May 6, 2010

(54) METHOD FOR PREVENTION OF TUMOR

(75) Inventors: Tomohiko Ohta, Tokyo (JP); Ko Sato, Kanagawa (JP)

Correspondence Address: DICKSTEIN SHAPIRO LLP, 1633 Broadway, NEW YORK, NY, 10019, US

(73) Assignee: St. Marianna University School of Medicine, Kanagawa (JP)

(21) Appl. No.: 12/097,443

(22) PCT Filed: Dec. 18, 2006
(86) PCT No.: PCT/JP2006/325679

§ 371 Date: Dec. 30, 2009

Related U.S. Application Data
(60) Provisional application No. 60/750,877, filed on Dec. 16, 2005.

(51) Int. Cl. A61K 31/7105; C12Q 1/68; C07H 21/02; A61P 35/00

(52) U.S. Cl. 514/44 A; 435/6; 536/24.5

(57) ABSTRACT

The present invention provides a method for inhibiting a tumor, which comprises suppressing the expression of HERC2. In one embodiment of the present invention, the suppression of HERC2 is induced by the expression of BRCA1.

### TECHNICAL FIELD

The present invention relates to a method for suppressing a tumor, which comprises suppressing the expression of HERC2. Moreover, the present invention relates to a method for suppressing a tumor in a cell or a tissue and a method for inducing antitumor activity in a cell or a tissue, which comprise inhibiting the binding of HERC2 with BRCA1.

### BACKGROUND OF THE INVENTION

HERC2 (hect domain and RCC-like domain 2) is a gene found in the hot spot of a deletion break point existing on human long arm of chromosome 15 (bands 15q11 and 15q13), and it is associated with the Prader-Willi/Angelman syndrome (PWS/AS)¹⁻⁵. The duplication of HERC2 in this chromosomal region is considered to be a crucial factor for increasing the incidence of homologous recombination errors in formation of sperms. In addition, by such duplication of HERC2, deletion occurs in a 4-Mb chromosomal region, and several genes including UBE3A that is a gene encoding E6-AP (a human papilloma virus E6-associated protein) are eliminated by such deletion. A maternal allele playing a role in protecting PWS falls into silencing during oogenesis or early embryonal formation in a process known as genome imprinting. Thus, PWS is caused only by the presence of the aforementioned deletion in a paternal chromosome. In some PWS patients, HERC2 is mutated. However, mutation of HERC2 itself is not a cause of PWS. This is because a HERC2 gene does not undergo genome imprinting and because only the expression of a maternal HERC2 allele is considered to be sufficient to avoid the symptoms of PWS⁶.

Nevertheless, homozygous mutation of this gene causes a variety of phenotypes in mice ^{I}. For example, rjs (renty jerkey sterile) and jdf2 (juvenile development and fertility⁸) have been known as phenotypes similar to the symptoms of PWS. Male phenotypes include sterility, testicular dysgenesis, failure of spermatogenesis and morphological defect of sperm. Female phenotypes include a diminished uterus size, and an ovary which comprises an overgrown ovarian follicle but in which corpus luteum or corpora hemorrhagica is hardly formed.

A HERC2 gene is composed of 93 exons and encodes a 528-kDa enormous protein consisting of 4834 amino acids^{1,2}.

The nucleotide sequence (SEQ ID NO: 1) of the HERC2 gene (AF071172) and the amino acid sequence (SEQ ID NO: 2) encoded by this gene are evolutionarily highly conserved. Human HERC2 shows a homology of 70% with drosophila HERC2 over 743 amino acids on the carboxy-terminal side³. This homology suggests that the HERC2 gene has an important cellular function. HERC2 has a large number of functional domains including 3 RLDs (RCC-like domains), a DOC domain, an M-H domain, a cytochrome b5-like domain, an ZZ-type zinc finger and a C-terminal HECT domain. Based on these domain structures, the present inventors have assumed that HERC2 would be associated with protein transport and decomposition via ubiquitin.

It has been considered that BRCA1, a suppressor of breast cancer and ovarian cancer factor, is associated with many nerve pathways for preventing tumor progression. For example, a BRCA1-deficient cell exhibits genome instability. This genome instability is considered to occur due to dysfunction of BRCA1⁹⁻¹¹. Such genome instability causes a decrease in the ability of

DNA damage repair, a decrease in transcriptional control mechanism, apoptosis induction, a decrease in an S-phase or G2-M checkpoint function, a decrease in the control of centrosome replication, etc. BRCA1 is phosphorylated by kinases of the ATM/ATR family after genotoxic stress^{12,13}. Thereafter, the phosphorylated BRCA1 binds to and cooperates with a DNA repair protein such as Rad51 or Rad50-Mre11-Nbs1, so that it performs the homologous recombination repair of the DNA damage site^{14,15}. On the other hand, the expression of BRCA1 is down-regulated in the late stage of DNA damage response^{16,17}. A main mechanism that plays a role in this down-regulation is transcriptional repression via p53¹⁶.

However, p-53-independent and proteasome-dependent protein decomposition is also associated with the down-regulation of BRCA1 induced by DNA damage¹⁸. A mechanism for causing such down-regulation has not yet been clarified so far.

### DISCLOSURE OF THE INVENTION

It is an object of the present invention to provide a method for suppressing tumor growth. As a result of intensive studies directed towards achieving the aforementioned object, the present inventors have found that such tumor growth can be suppressed by suppressing the expression of HERC2 in a cell, thereby completing the present invention.

Specifically, the present invention is as follows.

(1) A method for suppressing a tumor, which is characterized in that it comprises suppressing the expression of HERC2 in a cell.

In the method of the present invention, the expression of HERC2 is suppressed by siRNA that acts on a HERC2 gene, for example.

(2) A method for suppressing a tumor, which is characterized in that it comprises inhibiting the interaction between HERC2 and BRCA1 in a cell.

The interaction between HERC2 and BRCA1 is inhibited by at least one selected from the group consisting of siRNA acting on the HERC2 gene, a HERC2 inhibitor, and an antibody reacting with the HERC2, for example.

(³) A method for screening an antitumor agent, which is characterized in that it comprises measuring the expression level of HERC2 in a cell in the presence of a candidate substance and then selecting a substance having antitumor activity using the obtained measurement result as an indicator.

(4) A method for screening an antitumor agent, which is characterized in that it comprises measuring the interaction between HERC2 and BRCA1 in the presence of a candidate substance and then selecting a substance having antitumor activity using the obtained measurement result as an indicator.

(5) An antitumor agent comprising a substance that suppresses the expression of HERC2 in a cell.

An example of the substance that suppresses the expression of HERC2 is siRNA that acts on a HERC2 gene.

(6) An antitumor agent comprising a substance that inhibits the interaction between HERC2 and BRCA1 in a cell.

The substance that inhibits the interaction between HERC2 and BRCA1 is at least one selected from the group consisting of siRNA acting on the HERC gene, a HERC2 inhibitor, and an antibody reacting with the HERC2.

(7) A method for treating a tumor, which is characterized in that it comprises suppressing the expression of HERC2 in vivo.

(8) A method for treating a tumor, which is characterized in that it comprises inhibiting the interaction between HERC2 and BRCA1 in vivo.

(9) Use of a substance suppressing the expression of HERC2 in a cell for the production of a pharmaceutical for treating a tumor.

(10) Use of a substance inhibiting the interaction between HERC2 and BRCA1 in a cell for the production of a pharmaceutical for treating a tumor.

According to the present invention, the function of the HERC2 protein has been discovered for the first time. That is, it was found that HERC2 functionally interacts with BRCA1, so that it controls the stability of BRCA1 and the cellular localization of BRCA1. It was also found that since HERC2 is a protein that responds to DNA damage, if such DNA damage is induced, a BRCA1 protein is suppressed. These results demonstrate that a mechanism in which decomposition of the BRCA1 protein takes place after DNA damage has been clarified. It is said that this mechanism is important for determining the destiny of cells. The functional interaction of HERC2 with BRCA1 means that breast cancer and ovarian cancer are associated with Prader-Willi/Angelman syndrome in the background of molecular biology. Specifically, the suppression of protein decomposition via ubiquitin due to the deletion of UBE3A (ubiquitin ligase E3) contained in the structure of HERC2 is considered to be one of the causes of the Prader-Willi/Angelman syndrome. On the other hand, with regard to the down-regulation of the BRCA1 protein that is possibly associated with breast cancer and ovarian cancer, it was found in the present invention that HERC2 interacts with BRCA1, and thus, it was demonstrated that this down-regulation does not involve p53. Accordingly, it is considered that UBE3A contained in the structure of HERC2 is involved in PWS/AS.

### BRIEF DESCRIPTION OF THE DRAWINGS

FIG. 1 is a view showing HERC2 that has been identified as a protein contained in a BRCA1 immune complex as a result of a mass spectrometry screening.

FIG. 1A is a view showing a putative HERC2 protein and a putative functional domain thereof, as well as the positions of 10 peptides identified by

LC/MS/MS. The position of HERC2-CT (4254-4834) used in this test is also shown in the figure.

FIG. 1B is a view showing the interaction of the transfected HERC2-CT with BRCA1. 293T cells were transfected with a certain plasmid. Total cell lysates (lanes 1 to 3) or immune precipitates using anti-FLAG antibodies (lanes 4 to 6) and immune precipitates (lanes 7 to 9) using anti-Myc antibodies were subjected to immunoblotting using certain antibodies. IP: immune precipitate; IB: Immunoblotting

FIG. 2 is a view showing BRCA1 destabilizled by HERC2 in vivo.

With regard to FIG. 2A, 293T cells in a p100 plate were transfected with plasmids encoding FLAG-BRCA1 (1-772) (lanes 1-4: 10 μg) and gradually increasing amounts of Myc-HERC2-CT (lane 2: 1 μg; lane 3: 2 μg; lane 4: 5 μg). A parent pcDNA 3 vector was added to the cells, so that the total plasmid DNA amount was adjusted to be 15 μg per plate. The steady-state level of each protein was analyzed by immunoblotting using an anti-FLAG antibody, an anti-Myc antibody, or an anti-tubulin antibody.

FIG. 2B is a view showing the results obtained by analyzing the steady-state level of each protein in the same manner as that in FIG. 2A with the exception that endogenous BRCA1 was analyzed instead of FLAG-BRCA1.

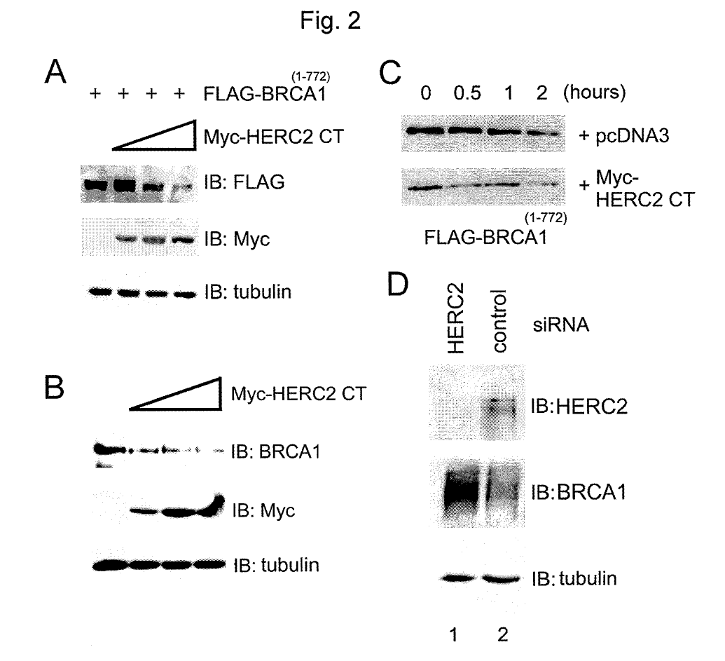

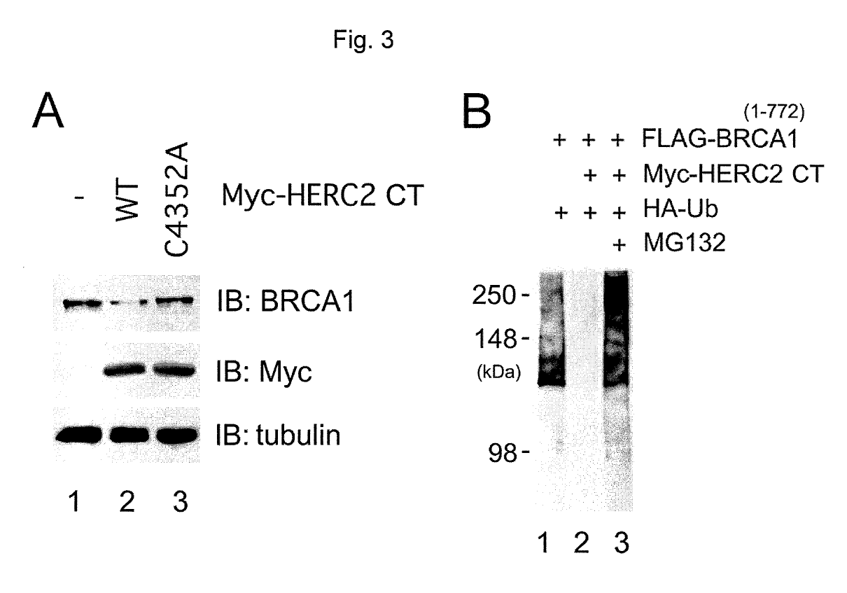

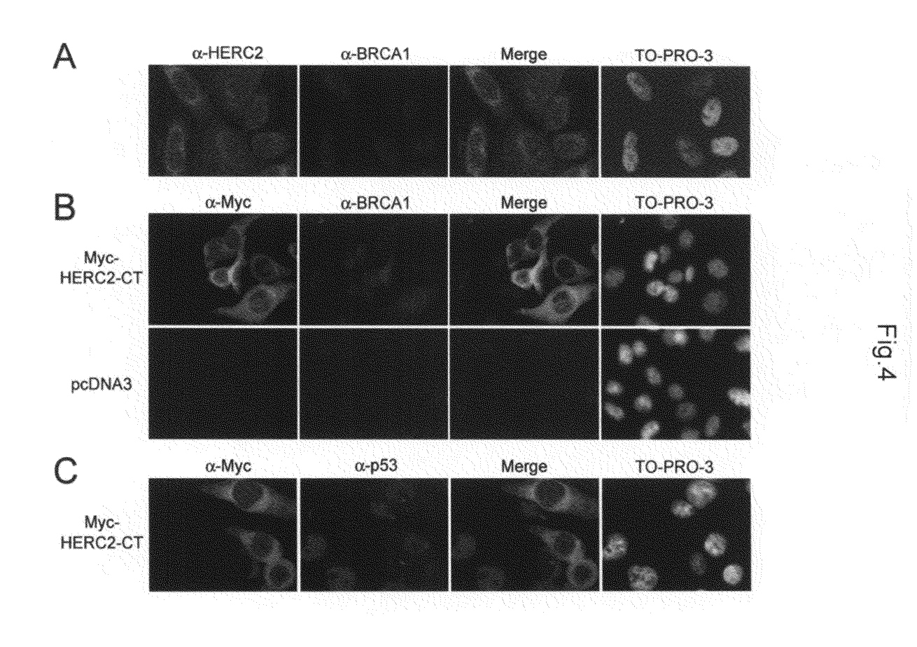

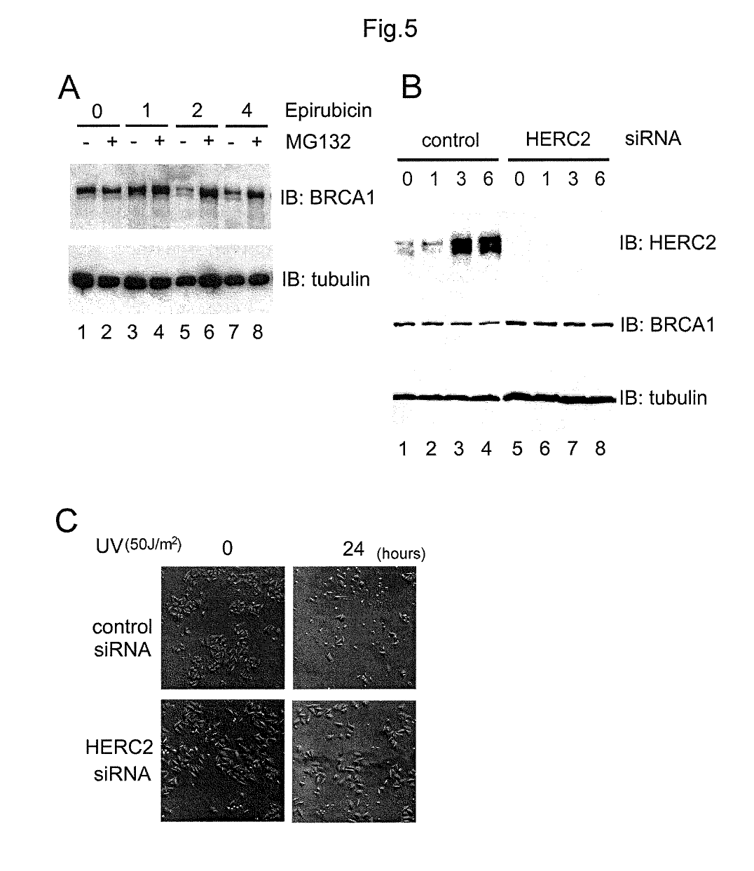

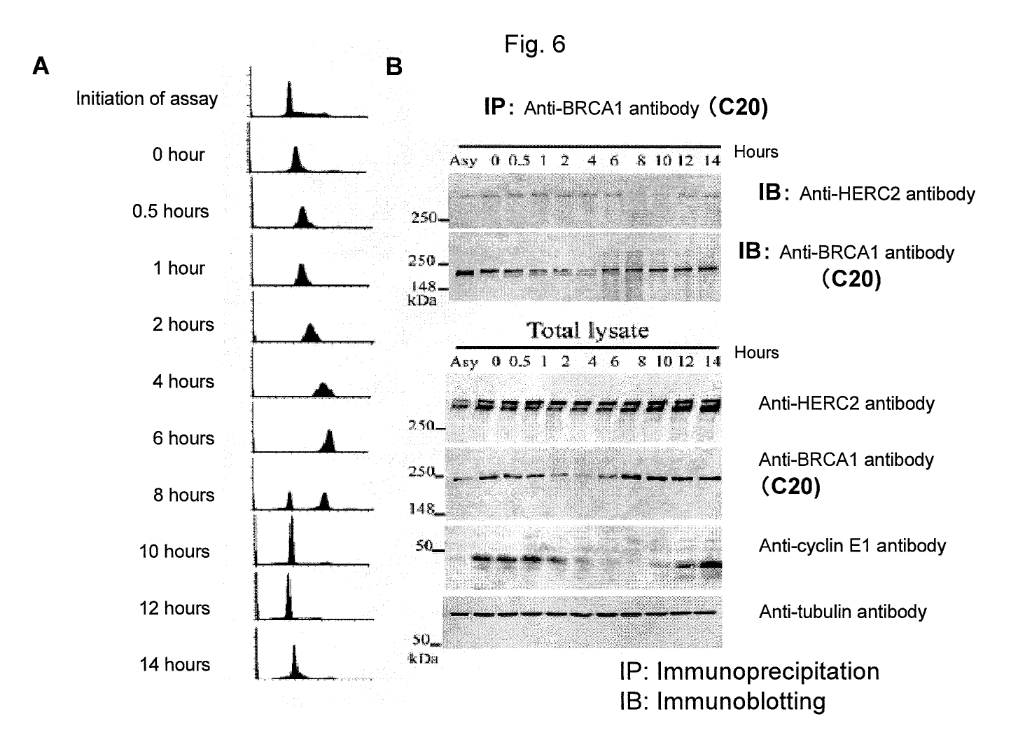

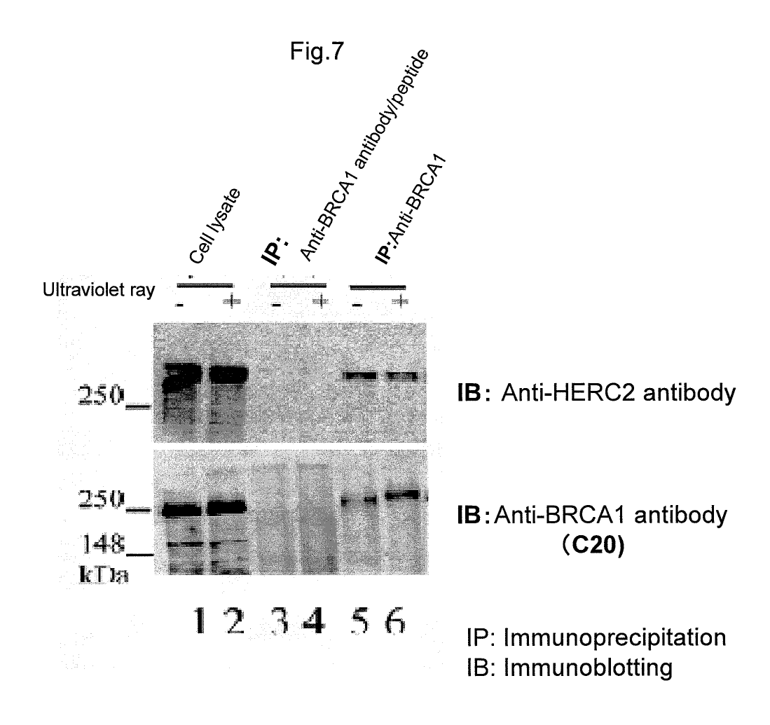

With regard to FIG. 2C, 293T cells were transfected with plasmids encoding FLAG-BRCA1¹⁻⁷⁷² and either a parent pcDNA3 vector or Myc-HERC2-CT. The cells were incubated with cycloheximide (10 μM), and they were then tracked for a certain period of time. Subsequently, a cell lysate was subjected to immunoblotting using an anti-FLAG antibody.

With regard to FIG. 2D, HeLa cells were transfected with HERC2-specific siRNA (lane 1) or control siRNA (lane 2). A cell lysate was subjected to 3%-8% gradient (upper and central panels) and 7.5% gradient (lower panel) SDS-PAGE. Subsequently, it was analyzed by immunoblotting using certain antibodies.

FIG. 3 is a view showing the potential involvement of HERC2 ubiquitin ligase activity in decomposition of BRCA1.

With regard to FIG. 3A, 293T cells were transfected with a plasmid expressing a wild type (lane 2) or the C4352A mutant of Myc-HERC2-CT. A total cell lysate was subjected to immunoblotting using certain antibodies.

With regard to FIG. 3B, 293T cells transfected with a certain plasmid were treated with 10 μM MG132 (lane 3) or a DMSO solvent (lanes 1 and 2) for 14 hours. They were then boiled in a 1% SDS-containing buffer, so that it was diluted to 0.1% SDS, followed by immunoprecipitation with anti-FLAG-antibody-crosslinked beads. FLAG-RPB8 was eluted with a

FLAG peptide, and 7.5% SDS-PAGE was then performed. Thereafter, the cells were analyzed by immunoblotting using an anti-HA antibody.

FIG. 4 is a view showing the cellular localization of BRCA1 generated by the excessive expression of HERC2-CT.

With regard to FIG. 4A, growing HeLa cells were fixed with 3% formalin, and a certain antibody was then added thereto. Subsequently, the cells were stained with a FITC (green) or rhodamine (red)-bound secondary antibody. The nucleus was stained with TO-PRO-3. The term “merge” means a photograph formed by overlapping two protein images.

Each of FIGS. 4B and 4C is a view showing the results obtained by transfecting 293T cells with either Myc-HERC2-CT or a parent pcDNA vector, and then staining the cells with a certain antibody described in FIG. 4A.

FIG. 5 is a view showing that the decomposition of BRCA1 after DNA damage is recovered by the knockdown of HERC2.

With regard to FIG. 5A, T47D cells were incubated with epirubicin (0.2 μg/ml) in the presence or absence of MG132 (50 μM) for a certain period of time. A total cell lysate was subjected to 7.5% SDS-PAGE, and it was then analyzed by immunoblotting using certain antibodies.

With regard to FIG. 5B, an ultraviolet ray (35 J/m²) was applied to HeLa cells transfected with control siRNA (lanes 1 to 4) or HERC2-specific siRNA (lanes 5 to 8), and the cells were then recovered at a certain point after completion of the ultraviolet irradiation. A cell lysate was subjected to 3%-8% gradient (upper panel) or 7.5% gradient (central and lower panels) SDS-PAGE. Subsequently, it was analyzed by immunoblotting using the anti-HERC2 antibody, anti-BRCA1 antibody or anti-tubulin antibody as shown in the figure.

With regard to FIG. 5C, HeLa cells were transfected with control siRNA (upper panel) or HERC2-specific siRNA (lower panel). Cell viability obtained before (left panel) or 24 hours after (right panel) the ultraviolet irradiation (50 J/m²) was observed by a phase-contrast microscopy method.

FIG. 6A is a view showing the synchronization of cell cycle.

FIG. 6B (columns 1 and 2) is a view showing the results of immunoprecipitation using an anti-BRCA1 antibody.

FIG. 6B (columns 3 to 6) is a view showing the results of immunoblotting performed on a total cell lysate.

FIG. 7 is a view showing a change in the interaction of HERC2 with BRCA1 due to ultraviolet irradiation.

### BEST MODE FOR CARRYING OUT THE INVENTION

Hereinafter, the present invention will be described in detail. All patent applications, patents, publications and websites cited herein are incorporated herein by reference in their entirety.

### 1. SUMMARY

HERC2 is a highly mutable, large gene, which is found in the hot spot of a deletion break point in the Prader-Willi/Angelman syndrome. This gene exists on human long arm of chromosome 15q11-q13. It has been known that if this gene is deleted, a variety of phenotypes, such as rjs and jdf2, are generated in mice.

The term “rjs” means “runty jerky sterile,” and it has a phenotype regarding developmental disability, tremor and azoospermia. On the other hand, the term “jdf2” means “juvenile development and fertility,” and it has a phenotype regarding developmental disability and infertility. These phenotypes are all similar to the symptoms of the Prader-Willi/Angelman syndrome.

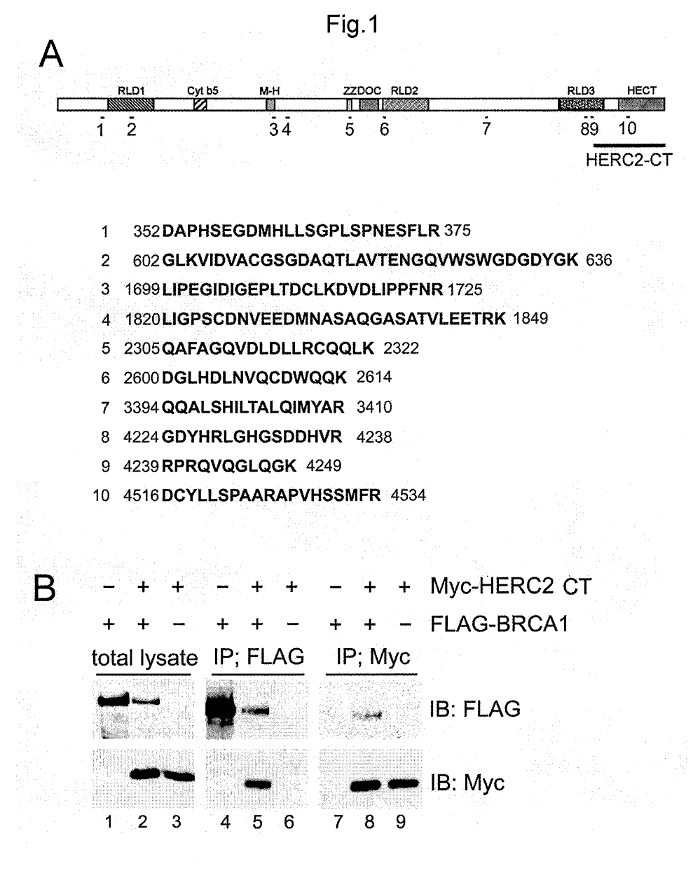

HERC2 has RLD and HECT domains (FIG. 1A). These domains are a sequence similar to RCC1 and ubiquitin ligase, each of which has GEF activity (activity of eliminating GDP from Ran and exchanging it with RanGTP). Thus, it is suggested that the RLD and HECT of HERC2 play a role in protein transport and decomposition pathway, respectively. However, their exact cellular functions are unknown.

The present invention has been completed based on the findings that, in response to DNA damage, HERC2 down-regulates BRCA1 acting as a suppressor of breast cancer and ovarian cancer. The present invention is characterized in that the expression of HERC2 is suppressed so as not to down-regulate BRCA1, or the interaction of HERC2 with BRCA1 is suppressed to retain the activity of BRCA1, thereby suppressing tumor.

In the present invention, in order to obtain an immune complex protein that binds to BRCA1, a screening was performed based on mass spectrometry. As a result, HERC2 was identified as a partner protein that specifically binds to BRCA1. There was produced a C-terminal fragment (hereinafter referred to as HERC2-CT) consisting of the amino acid sequence (SEQ ID NO: 3) of amino acids at positions 4254-4834 from the amino acid sequence (SEQ ID NO: 2) of HERC2, which comprises a HECT domain in this fragment region. Thereafter, the interaction of HERC2-CT with BRCA1 was analyzed. As a result, it was found that HERC2-CT interacted with BRCA1 in vivo, so that the HERC2-CT induced the decomposition of the BRCA1. In contrast, in a case where HERC2 was knocked-down by siRNA, BRCA1 was stabilized.

HERC2 is mainly localized in a cytoplasm. However, when HERC2-CT was excessively expressed, interestingly, the localization of BRCA1 to such a cytoplasm was induced. This phenomenon suggests that HERC2 may trap BRCA1 in a cytoplasm.

Moreover, the present inventors have found that HERC2 is a DNA damage responsive protein, that the expression of HERC2 is sharply increased 3 to 6 hours after ultraviolet irradiation, and that an increase in the expression of HERC2 occurs due to the regression of a BRCA1 protein.

Furthermore, when HERC2 was knocked-down, BRCA1 was recovered to a normal level, and the cells became resistant to ultraviolet irradiation. This ultraviolet resistance is a concept that is in contrast with ultraviolet hypersensitivity induced by the deletion of BRCA1. Thus, these results determine the cellular function of HERC2 as a gene product associated with the Prader-Willi/Angelman syndrome, proposing a concept that such function is associated with breast cancer and ovarian cancer in the background of molecular biology.

### 2. Inhibition of Expression and Activity of HERC2

In order to enhance the activity of BRCA1, a method for inhibiting the expression of HERC2 is adopted in the present invention.

A method for inhibiting the expression of HERC2 is not particularly limited. For example, RNA interference (RNAi) may be used. That is, siRNA (small interfering RNA) acting on a HERC2 gene was designed and synthesized, and the thus synthesized siRNA is then introduced into a cell, thereby causing RNAi.

RNAi is a phenomenon whereby dsRNA (double-stranded RNA) specifically and selectively binds to a target gene and cleaves it, so as to efficiently inhibit the expression thereof. For example, if dsRNA is introduced into a cell, the expression of a gene having a sequence homologous to the RNA is suppressed (knocked-down).

siRNA is designed as follows.

(a) The used gene is not particularly limited, as long as it is a gene encoding HERC2. Any given region can be used as a candidate. For example, in the case of a human, any given region with GenBank Accession No. AF071172 (SEQ ID NO: 1) can be used as a candidate.

(b) A sequence beginning with AA is selected from the selected region. The length of the sequence is 19 to 25 nucleotides, and preferably 19 to 21 nucleotides. The sequence may be selected such that the GC content thereof can be 40% to 60%, for example.

In order to introduce siRNA into a cell, a method of ligating siRNA synthesized in vitro to plasmid DNA and then introducing the thus ligated product into a cell, a method of annealing two RNA portions, etc. can be applied.

Moreover, in order to obtain the RNAi effect, shRNA can also be used in the present invention. The shRNA is also referred to as short hairpin RNA, and it is an RNA molecule having a stem-loop structure that is a pattern obtained when parts of regions of a single strand form a complementary strand.

Such shRNA can be designed such that a portion thereof forms a stem-loop structure. For example, the sequence of a certain region is defined as sequence A, and a strand complementary to such sequence A is defined as sequence B. Sequence A, a spacer, and sequence B are arranged such that these sequences can exist on a single RNA strand in this order, and such that the entire length can be 45 to 60 nucleotides. Sequence A is the sequence of a region of the HERC2 gene (SEQ ID NO: 1) used as a target. Such a target region is not particularly limited, and any given region can be a candidate.

The length of sequence A is 19 to 25 nucleotides, and preferably 19 to 21 nucleotides.

3. Inhibition of Interaction of HERC2 with BRCA1

The present invention provides an antitumor method, which is characterized in that it comprises inhibiting the interaction of HERC2 with BRCA1 in a cell.

The interaction of HERC2 with BRCA1 can be inhibited using siRNA that acts on a HERC gene, a HERC2 inhibitor, an antibody reacting with HERC2, etc. With the use of these components, the function of HERC2 to interact with BRCA1 is lost, and the activity of BRCA1 is thereby maintained or increased, so that antitumor activity is induced.

Such siRNA acting on the HERC2 gene can be designed as described above.

An example of such a HERC2 inhibitor is a ubiquitination inhibitor, but examples are not limited thereto.

The aforementioned antibody reacting with HERC2 means an immunoglobulin that recognizes the entire or a part of HERC2 and specifically binds to the recognized site, so as to decrease or remove the activity of the HERC2. A method for producing such an antibody is known to persons skilled in the art.

### 4. Screening Method

The screening method of the present invention is characterized in that it comprises measuring the expression level of HERC2 in a cell in the presence of a candidate substance and then selecting a substance having antitumor activity using the obtained measurement result as an indicator.

In addition, the present invention provides a method for screening an antitumor agent, which is characterized in that it comprises measuring the interaction of HERC2 with BRCA1 in the presence of a candidate substance and then selecting a substance having antitumor activity using the obtained measurement result as an indicator.

A candidate substance means a test substance, which is to be subjected to screening for the use as an antitumor agent, and any given substance can be used as such a candidate substance. The type of a candidate substance is not particularly limited. Examples of such a candidate substance include a peptide, a protein, a nonpeptide compound, and a synthetic compound (a high molecular weight or low molecular weight compound). Other examples of such a candidate substance also include natural products and extracts such as fermented products, cell extracts, cell culture supernatants, plant extracts, tissue extracts of mammals (e.g. a mouse, a rat, a swine, a bovine, a sheep, a monkey, a human, etc.), and blood plasma. These compounds may be either novel compounds or known compounds. Further, a candidate substance may form a salt. Examples of the salt of a candidate substance used herein include salts with a physiologically acceptable acid (e.g. inorganic acid, organic acid, etc.), a nucleotide (e.g. metallic acid, etc.), and the like. Moreover, a compound library, a phage display library, and a combinatorial library may also be used. A compound library may be constructed by known means. Alternatively, a commercially available compound library may also be used.

In the screening method of the present invention, the expression of HERC2 or the interaction of HERC2 with BRCA1 is measured in the presence of a candidate substance (a test substance). For example, a candidate substance of an antitumor agent is allowed to come into contact with a cell, so that the expression level of HERC2 or the binding of HERC2 with BRCA1 in a cell can be measured. The term “come into contact with” means that a cell and a candidate substance are allowed to exist in a single reaction system or culture system. Thus, the term “come into contact with” includes the addition of a candidate substance to a cell culture vessel, the mixing of a cell with a candidate substance, and the culture of a cell in the presence of a candidate substance.

A method for measuring the expression level of HERC2 and a method for measuring the interaction of HERC2 with BRCA1 are not particularly limited. The aforementioned interaction itself may be directly measured, or by measuring the activity of BRCA1 or HERC2, the aforementioned interaction may be indirectly measured. Examples of a method for measuring interaction include an RT-PCR method, Northern blotting, an immunoprecipitation method, a pull-down assay method, Western blotting, NMR, surface plasmon resonance (SPR), gel shift assay, and gel filtration. In general, it is preferable that the same assay system be carried out also in the absence of a candidate substance, that the expression level of HERC2 or the aforementioned interaction be measured in both cases, namely, in the presence of a candidate substance and in the absence of a candidate substance, and that the two results be compared with each other, so as to determine whether or not the candidate substance inhibits the expression of HERC2 or the aforementioned interaction.

### 5. Pharmaceutical Composition

In the present invention, a substance that suppresses the expression of HERC2 in a cell and a substance that inhibits the interaction of HERC2 with BRCA1 in a cell can be used as antitumor agents. In particular, since siRNA and shRNA produced to suppress the expression of HERC2, a HERC inhibitor, and an antibody reacting with HERC2 suppress the expression of HERC2, these components can be particularly used as pharmaceutical compositions in the gene therapy of tumors.

The pharmaceutical composition of the present invention can be used as an antitumor agent by administering it into a living body. Further, in order to produce the aforementioned pharmaceutical composition, a substance that suppresses the expression of HERC2 in a cell or a substance that inhibits the interaction of HERC2 with BRCA1 in a cell can be used.

A tumor site, to which the present pharmaceutical composition is applied, is not particularly limited. Examples include brain tumor, tongue cancer, pharyngeal cancer, lung cancer, breast cancer, esophageal cancer, stomach cancer, pancreatic cancer, biliary tract cancer, gallbladder cancer, duodenal cancer, colon cancer, liver cancer, uterine cancer, ovarian cancer, prostatic cancer, renal cancer, bladder cancer, rhabdomyosarcoma, fibrosarcoma, osteosarcoma, chondrosarcoma, skin cancer, and various types of leukemia (for example, acute myelocytic leukemia, acute lymphatic leukemia, chronic myelocytic leukemia, chronic lymphatic leukemia, adult T-cell leukemia, and malignant lymphoma). The aforementioned tumors may be primary lesions, metastatic focuses, or tumors occurring with other diseases.

The pharmaceutical composition of the present invention may adopt either a dosage form for oral administration or a dosage form for parenteral administration. In the case of parenteral administration, it is also possible to directly administer the pharmaceutical composition to the aforementioned tumor sites.

These dosage forms can be formulated according to ordinary methods. The pharmaceutical composition may comprise a pharmaceutically acceptable carrier and an additive. Examples of such a carrier and an additive include water, a pharmaceutically acceptable organic solvent, collagen, polyvinyl alcohol, polyvinylpyrrolidone, a carboxyvinyl polymer, carboxymethylcellulose sodium, sodium polyacrylate, sodium alginate, water soluble dextran, carboxymethyl starch sodium, pectin, methylcellulose, ethylcellulose, xanthan gum, gum Arabic, casein, agar, polyethylene glycol, diglycerine, glycerine, propylene glycol, Vaseline, paraffin, stearyl alcohol, stearic acid, human serum albumin, mannitol, sorbitol, lactose, and a surfactant that is acceptable as a pharmaceutical additive.

Such additives are selected from the aforementioned substances, and they are then used singly or by appropriately combining them, depending on the dosage form of the antitumor agent of the present invention. In the case of oral administration, examples of a suitable dosage form include a tablet, a capsule, a parvule, a powder, a granule, a liquid preparation, a syrup, and other appropriate dosage forms. In the case of parenteral administration, examples of a suitable dosage form include a transpulmonary agent-type (for example, the use of a nebulizer), a transnasal agent-type, a transdermal agent-type (for example, an ointment and a cream), and an injection-type. In the case of an injection-type, the pharmaceutical composition of the present invention can be systemically or topically administered via an intravenous injection such as a drop, an intramuscular injection, an intraperitoneal injection, a hypodermic injection, etc.

For example, when the present pharmaceutical composition is used as an injectable formulation, a substance that suppresses the expression of HERC2 or a substance that inhibits the interaction of HERC2 with BRCA1 is dissolved in a solvent (for example, a normal saline, a buffer, a glucose solution, etc.). Thereafter, Tween 80, Tween 20, gelatin, human serum albumin, etc. are added to the solution, and the obtained solution can be then used. Alternatively, the pharmaceutical composition is freeze-dried to prepare a dosage form that can be thawed before use. Examples of an excipient used in freeze-drying include sugar alcohols and sugars such as mannitol or glucose.

The dose of the pharmaceutical composition of the present invention differs depending on age, sex, symptoms, an administration route, the number of doses, and a dosage form. The administration method is selected, as appropriate, depending on the age of a patient and symptoms. An effective dose is 0.01 μg to 1,000 mg, and preferably 0.1 μg to 100 μg per administration per kg of body weight. However, the dose of the aforementioned therapeutic agent is not limited to the above range.

When the pharmaceutical composition of the present invention is used as an agent in the gene therapy of tumors, a site to which the pharmaceutical composition is applied is not particularly limited, and the aforementioned tumor sites may be exemplified. The aforementioned tumors may be primary lesions, metastatic focuses, or tumors occurring with other diseases.

When the pharmaceutical composition of the present invention is used as an agent in gene therapy, a method of directly administering the pharmaceutical composition of the present invention in the form of an injection and a method of administering a vector into which a nucleic acid has been incorporated may be applied. Examples of the aforementioned vector include an adenovirus vector, an adeno-associated virus vector, a herpesvirus vector, a vaccinia virus vector, a retrovirus vector, and a lentivirus vector. Using these vectors, the pharmaceutical composition of the present invention can be efficiently administered.

Moreover, it is also possible to introduce the pharmaceutical composition of the present invention into a phospholipid vesicle such as liposome and to administer the resultant vesicle to a patient. A vesicle that contains siRNA or shRNA is introduced into a certain cell by a lipofection method. Thereafter, the obtained cell is systemically administered into vein, artery, etc. It can also be topically administered to a tumor site, etc.

The dose of the pharmaceutical composition of the present invention differs depending on age, sex, symptoms, an administration route, the number of doses, and a dosage form. In the case of adenovirus, for example, the dose is approximately 10⁶ to 10¹³ cells, once a day, at intervals of 1 to 8 weeks.

In order to introduce siRNA or shRNA into a tissue or organ of interest, a commercially available gene introduction kit (for example, AdenoExpress manufactured by Clontech) can be used.

Hereinafter, the present invention will be more specifically described in the following examples. However, these examples are not intended to limit the scope of the present invention.

### EXAMPLES

Mass Spectrometry

The transfected 293T cells were incubated with 120 μl-volume anti-FLAG-antibody-crosslinked beads (Sigma) in 50 μM MG132 for 10 hours. Thereafter, a protein interacting with FLAG-BRCA1 was immunoprecipitated from the transfected 293T cells contained in two p150 plates. The protein was eluted from the beads in 60 μl of 25 mM ammonium bicarbonate that contained 0.1 mg/ml FLAG peptide, and it was then digested with 7.4 μg/ml trypsin at 30° C. for 20 hours. Subsequently, a peptide fragment was subjected to LC/MS/MS according to a known method¹⁹. Using Mascot software program (Matrix Science, London, UK), the protein database of the National Center for Biotechnology Information (NCBI) was searched, and the acquired collisional dissociation spectrum was then analyzed.

Plasmid

cDNA corresponding to the C-terminus (4254-4834) of human HERC2 was amplified by PCR. The cDNA library of MCF10A cells was used as a template. Primers having the following nucleotide sequences were used:

(SEQ ID NO: 4)

F primer: TAGGATCCCCTTACCAAATCTGGAGC
(underlined portion: BamHI site);
and

(SEQ ID NO: 5)

R primer: TAGCTCTCATCTCTCGAGGACGTTTC
(underlined portion: XhoI site).

The composition of a reaction solution and reaction conditions are as follows (in the case of using an instruction for use).

<Composition of reaction solution>

| | | |
|---|---|---|
| Template (50 ng/μl): | 1 | μl |
| Pfx buffer: | 2 | μl |
| (containing dNTP, AccuPrime ™ protein, and MgSO₄) | | |
| Pfx polymerase (Stratagene): | 0.4 | μl |
| F primer (100 pmol/μl): | 0.6 | μl |
| R Primer (100 pmol/μl): | 0.6 | μl |
| Sterilized water: | 15.4 | μl |
| Total: | 20 | μl |

### <Reaction Conditions>

After a heating process at 95° C. for 2 minutes, 1 cycle consisting of heat denaturation at 95° C. for 15 seconds, annealing at 50° C. for 30 seconds and an elongation reaction at 68° C. for 1 minute was repeated for 25 cycles in total. Thereafter, the resultant was incubated at 72° C. for 10 minutes, and it was then cooled at 4° C.

A fragment obtained after amplification and an N-terminal Myc tag were subcloned in frame into a pcDNA3 vector. As mammalian expression plasmids for BRCA1, BARD1 and ubiquitin, known plasmids were used^{19,29}. FLAG-BRCA1 was provided from Dr. Richard Baer (Columbia University). C4352A, a HERC2 mutant, was produced using a site-directed mutagenesis kit (Stratagene). All the plasmids used were confirmed by DNA sequencing.

Cell Culture and Transfection

Cells (HeLa cells, etc.) were cultured in 5% CO₂ at 37° C., using a Dalbecco's Modified Eagle's Medium (DMEM), to which 10% fetal bovine serum and 1% antimicrobial-antifungal agent (Life Technologies, Inc. or Invitrogen) had been added. In order to measure the control of protein metabolic turnover by proteasome, a certain concentration of MG132 or a DMSO solvent having the same volume was added to the cells for a certain period of time. Thereafter, the cells were recovered. In order to analyze the half-life of a protein in vivo, the cells were incubated with 10 μg/ml cycloheximide (Wako) for a certain period of time. By applying a standard calcium phosphate precipitation method, 293T cells were transfected. In each transfection, a parent pcDNA3 vector was added, so that the total amount of plasmid DNA was controlled. With regard to an ultraviolet irradiation test, the cells were washed with PBS, and an ultraviolet ray having a certain dose such as 35 J/m² (254 nm; UVP Inc, Upland, Calif.) was then applied to the cells. The cells were allowed to grow in a fresh medium for various periods of times (for example, 3 hours). The cell survival rate was analyzed by a phase-contrast microscopy method or a trypan blue exclusion measurement method.

Cell Synchronization

Cell synchronization was carried out by a double thymidine block method.

Asynchronous HeLa cells were cultured in the presence of 2 mM thymidine for 18 hours, and they were then cultured in a medium, from which such thymidine had been removed, for 9 hours. Subsequently, the cells were cultured in the presence of 2 mM thymidine for 17 hours. Thereafter, the cells were transferred into a fresh medium, and they were then recovered at a predetermined time. The cells were stained with Propidium iodide, and the progression of the cell cycle was then monitored by flow cytometry using FACSCalibur (Becton Dickinson).

Antibody

A commercially available mouse monoclonal antibody reacting with HA (12CA5, Boehringer, Mannheim), Myc (9E10, BabCo), FLAG (M2, Sigma), α- or β-tubulin (DMIA+BMIB, Neomarkers), or HERC2 (BD Bioscience); a rabbit polyclonal antibody reacting with BRCA1 (C20 or sc-642, Santa Cruz), p53 (Cell Signaling Technology), or HERC2 (BD Bioscience); and a blocking peptide (sc-642P, Santa Cruz) of BRCA1 were purchased.

siRNA

A SMART pool (registered trade mark) HERC2 siRNA mixture and a control siRNA mixture were purchased from Dharmacon Research, Inc. The cells were transfected with double-stranded RNA (final concentration: 100 nM) using Oligofectamine (registered trade mark) (Invitrogen) in accordance with an instruction for use.

Immunoprecipitation Method and Immunoblotting

A ubiquitination substrate was detected in vivo, using a boiled buffer that contained 1% SDS, according to a known immunoprecipitation method and immunoblotting^{19, 30}.

Cells were dissolved in a 0.5% NP-40-containing buffer (50 mM Tris-HCl (pH 7.5), 150 mM NaCl, 0.5% Nonidet P-40, 50 mM NaF, 1 mM dithiothreitol (DTT), 1 mM NaVO₃, 1 mM PMSF and a protein inhibitor mixed solution). The cells were mixed with the buffer by rotating the solution at 4° C. for 30 minutes. Thereafter, the solution was centrifuged at 16,000 g at 4° C. for 10 minutes. A supernatant was recovered as a cell lysate, and it was then used in immunoprecipitation and immunoblotting.

For the immunoprecipitation of a BRCA1 complex, the cell lysate was allowed to react with an anti-BRCA1 antibody at 4° C. for 1 hour. Thereafter, a precipitation reaction was carried out at 4° C. for 1 hour using Protein A and G sepharose (Invitrogen). The precipitate was washed with a 0.5% NP-40 buffer 3 times, and it was then re-suspended in an SDS sample buffer (50 mM Tris-HCl (pH6.8), 0.2% bromophenol blue, 10% glycerol, 2% SDS, and 100 mM DTT). The suspension was boiled for 3 minutes. As a peptide competition control, an equal amount of blocking peptide was added.

HERC2 was detected by separating a sample in 3% to 8% NuPAGE (registered trade mark) gel (Invitrogen) by electrophoresis and then transferring it to a nitrocellulose membrane using XCell II (trade mark) Blot

Module (Invitrogen) in accordance with an instruction for use. BRCA1 and α- and β-tubulin were detected by separating a sample from 7.5% SDS-polyacrylamide gel and then transferring it to a nitrocellulose membrane using a Semi-dry transfer unit (Amersham Biosciences).

The membrane, to which each protein had been transferred, was incubated using a certain antibody. Thereafter, it was developed using an ECL Western blotting detection system (Amersham Biosciences), and it was then visualized using a Fuji LAS-3000 CCD camera.

Indirect Immunocytochemistry

Cells were fixed with 4% formalin for 15 minutes, and they were then permeabilized with 0.2% Triton X-100 for 5 minutes. Thereafter, the cells were washed with a phosphate buffered saline (PBS), and they were then blocked by 0.5% BSA in PBS, so that they were stained with a certain antibody. Primary antibodies were diluted with a blocking buffer in each of the following concentrations.

Anti-HERC2 antibody: 1 μg/ml

Anti-Myc antibody: 2 μg/ml

Anti-BRCA1 antibody: 2 μg/ml

An FITC- or rhodamine-bound secondary antibody (Jackson Immunosearch) was diluted at a dilution ratio of 1:50 for use. A nucleus was counterstained with 0.5 μM TO-PRO-3 (Molecular Probe). Subsequently, cells were encapsulated with a fluorescence encapsulating agent (BioLad), and they were then examined with a confocal laser scanning microscope (LSM510, Carl Zeiss).

### Example 1

Identification of HERC2 as Protein Interacting with BRCA1

In order to identify a protein that influences on the stability of BRCA1, the present inventors conducted nanoscale capillary liquid chromatography-tandem mass spectrometry (LC/MS/MS), so as to analyze a BRCA1 immune complex obtained from cells treated with MG132 used as a proteasome inhibitor. Among the thus identified proteins, HERC2 had a Mowse score of 80, and 10 types of peptides were considered to be highest.

The 10 types of peptides are as shown below (FIG. 1A).

(SEQ ID NO: 6)

| | | |
|---|---|---|
| 1. | 352-375: | DAPHSEGDMHLLSGPLSPNESFLR |

(SEQ ID NO: 7)

| | | |
|---|---|---|
| 2. | 602-636: | GLKVIDVACGSGDAQTLAVTENGQVWSWGDGDYGK |

(SEQ ID NO: 8)

| | | |
|---|---|---|
| 3. | 1699-1725: | LIPEGIDIGEPLTDCLKDVDLIPPFNR |

(SEQ ID NO: 9)

| | | |
|---|---|---|
| 4. | 1820-1849: | LIGPSCDNVEEDMNASAQGASATVLEETRK |

(SEQ ID NO: 10)

| | | |
|---|---|---|
| 5. | 2305-2322: | QAFAGQVDLDLLRCQQLK |

(SEQ ID NO: 11)

| | | |
|---|---|---|
| 6. | 2600-2614: | DGLHDLNVQCDWQQK |

(SEQ ID NO: 12)

| | | |
|---|---|---|
| 7. | 3394-3410: | QQALSHILTALQIMYAR |

(SEQ ID NO: 13)

| | | |
|---|---|---|
| 8. | 4224-4238: | GDYHRLGHGSDDHVR |

(SEQ ID NO: 14)

| | | |
|---|---|---|
| 9. | 4239-4249: | RPRQVQGLQGK |

(SEQ ID NO: 15)

| | | |
|---|---|---|
| 10. | 4516-4534: | DCYLLSPAARAPVHSSMFR |

HERC2 has a HECT domain at the C-terminus thereof. Thus, the present inventors assumed that HERC2 would be ubiquitin ligase associated with the decomposition of BRCA1, and they focused on HERC2 and further proceeded with the analysis.

The interaction of HERC2 with BRCA1 in vivo was confirmed by transient transfection and the subsequent immunoprecipitation (IP)-Western analysis. HERC2 is a significantly large protein consisting of 4834 amino acids (FIG. 1A). Hence, on the assumption that the C-terminal fragment region (4254-4834) (HERC2-CT) of HERC2 having a HECT domain plays a role in the binding and decomposition of BRCA1, the present inventors cloned the HERC2-CT. 293T cells were co-transfected with FLAG-BRCA1 and Myc-HERC2-CT, and Myc-HERC2-CT was then co-precipitated with FLAG-BRCA1 (FIG. 1B, lane 5). The FLAG-BRCA1 was detected in an anti-Myc-HERC2-CT immune precipitate (lane 8). This result demonstrates that the C-terminus of HERC2 interacts with BRCA1.

### Example 2

In vivo Destabilization of BRCA1 by HERC2

The present inventors have found that the expression of BRCA1 is significantly decreased in the co-expression of HERC2-CT (FIG. 1B, lanes 2 and 5). Thus, the present inventors analyzed whether or not HERC2 destabilizes BRCA1 in vivo. The steady-state level of BRCA1 and the half-life of a BRCA1 protein were analyzed in the presence and absence of Myc-HERC2-CT co-expression. The steady-state level of FLAG-BRCA1 (FIG. 2A) and that of endogenous BRCA1 (FIG. 2B) were dose-dependently decreased in the co-expression of HERC2-CT. It was demonstrated that when the cells were treated with cycloheximide also, the half-life of BRCA1 protein was decreased in vivo by HERC2-CT (FIG. 2C). Moreover, when HERC2 siRNA was introduced, the level of the endogenous BRCA1 protein was increased (FIG. 2D). From these results, it was revealed that HERC2 destabilizes BRCA1 in vivo. These results demonstrate that if the expression of HERC2 is suppressed, BRCA1 is activated and is able to suppress cancer.

### Example 3

### Potential Involvement of Ubiquitin Ligase Activity in Decomposition of BRCA1 via HERC2

HERC2-CT comprises a HECT domain. Thus, the present inventors assumed that the decomposition of a BRCA1 protein would be caused by the ubiquitin ligase activity of this motif. In order to examine the validity of this assumption, 293T cells were transfected with wild-type HERC2-CT or C4352A. C4352A is a mutant formed by substituting cysteine at position 4352 with alanine in the amino acid sequence of HERC2, and this mutant is characterized in that it is able to prevent E2 from binding to a HECT motif but in that it is not able to prevent the interaction with BRCA1.

With regard to an endogenous BRCA1 expression level, the action of wild-type HERC2-CT or C4352A was studied. As a result, it was found that the wild-type HERC2-CT decreased the expression of BRCA1, but that the C4352A mutant did not decrease such expression (FIG. 3A). Thereafter, the present inventors tried to confirm that BRCA1 was ubiquitinated in vivo by HERC2.

293T cells were co-transfected with FLAG-BRCA1 (1-772), Myc-HERC2-CT and HA-ubiquitin. It is clear that BRCA1 itself was self-ubiquitinated in vivo, although such a ubiquitination level was very low (FIG. 3B, lane 1). It is considered that this self ubiquitination was generated as a result of the interaction with endogenous BARD1. Interestingly, the ubiquitination of BRCA1 was drastically decreased by the co-expression of Myc-HERC2 (FIG. 3B, lane 2), but it was recovered by MG132 (FIG. 3B, lane 3). Self ubiquitination catalyzed by BRCA1-BARD1 is relevant to Lys6 of ubiquitin, and thus it is not enhanced in vivo by the addition of a proteasome inhibitor such as MG132 or LLnL¹⁹. Accordingly, it is considered that ubiquitination detected during the co-expression of Myc-HERC2-CT and exposure to MG132 is not associated with Lys6, but rather suggests that HERC2 mediates the polyubiquitination of BRCA1, acting as a signal for the decomposition of proteasome.

### Example 4

### Induction of Cellular Localization of BRCA1 by HERC2-CT Expression

BRCA1 shuttles between a nucleus and a cytoplasm, but it is mainly localized in the nucleus during interkinesis. On the other hand, the present inventors confirmed that HERC2 is predominantly localized in the cytoplasm (FIG. 4A). The transfected HERC2-CT is also localized in the cytoplasm

(FIGS. 4B and 4C). The transfected HERC2-CT interacts with BRCA1. Thus, the present inventors assumed that HERC2-CT might influence on the localization of BRCA1. Hence, in order to examine the validity of this assumption, the present inventors allowed HERC2-CT to transiently express in 293T cells, and thereafter, they analyzed the localization of BRCA1 by immunofluorescence microscopy.

As a result, it was found that almost all the cells transfected with a parent pcDNA vector had BRCA1 in the nucleus thereof (FIG. 4B, lower panel), but that BRCA1 existed both in the nucleus and in the cytoplasm in the case of cells that excessively expressed HERC2-CT (FIG. 4B; 6 cells found in the upper panel). These results demonstrate that HERC2-CT is able to enhance the extranuclear transport signal of BRCA1. Otherwise, it is considered that HERC2-CT suppresses the intranuclear transport signal of BRCA1.

Herein, a change in BRCA1 localization is not caused by direct action on BRCA1, but it is likely to generate as a result of the elimination of a general transport mechanism such as the elimination of importin/exportin activity. Thus, the present inventors analyzed the action of HERC2-CT on p53 that is a known importin/exportin regulatory cell protein.

As a result, the expression of HERC2-CT did not influence on the cellular localization of p53 (FIG. 4C). Accordingly, the present inventors concluded that the HERC2-CT-dependent cytoplasm localization of BRCA1 is not caused by deficiency in a general transport mechanism.

### Example 5

HERC2-dependent Down-regulation of BRCA1 after Ultraviolet Irradiation

Next, the present inventors studied whether or not the HERC2-mediated down-regulation of BRCA1 has a physiological influence. It has been widely known that the expression of BRCA1 is decreased after cells have undergone DNA damage. Such decrease is mainly caused by the p53-dependent transcription suppression of BRCA1^{16, 17}. However, it has also been reported that the decomposition of a BRCA1 protein also contributes to such decrease¹⁶⁻¹⁸. To date, p53-dependent BRCA1 decomposition has been previously reported. In the present example, it was confirmed that the BRCA1 protein level was down-regulated after DNA damage even in cells that had lost the function of p53, such as T47D or Hela cells (FIGS. 5A and 5B). This down-regulation is suppressed by MG132 acting as a proteasome inhibitor. Thus, it was suggested that decomposition by proteasome would be a cause of a partial decrease in BRCA1 expression (FIG. 5A).

Interestingly, the present inventors confirmed that HERC2 is drastically up-regulated after ultraviolet irradiation (FIG. 5B). Based on these findings, the present inventors analyzed whether or not HERC2 is involved in UV-ray-inducible BRCA1 decomposition.

siRNA was used to eliminate the expression of HERC2 from HeLa cells. Forty-eight hours later, ultraviolet ray (35 J/m²) was applied to the cells, and thereafter, the cells were recovered at several time points. The cells transfected with siRNA succeeded in the silencing of the expression of HERC2 (FIG. 5B, upper panel, lanes 5 to 8). On the other hand, in control cells, HERC2 was up-regulated 3 to 6 hours after the ultraviolet irradiation (lanes 3 and 4), and with such up-regulation, the suppression of BRCA1 was confirmed (central panel, lanes 1 to 4). An important point is that BRCA1 expression was not changed after the ultraviolet irradiation in cells in which the expression of HERC2 was suppressed (lanes 5 to 8). These results strongly suggest that BRCA1 is HERC2-dependently decomposed after the ultraviolet irradiation.

### Example 6

### Insensitivity of Cells to DNA Damage Caused by Ultraviolet Ray Due to HERC2 Knock-down

It has been widely known that if BRCA1 is deleted, cells become sensitive to DNA damage²⁰⁻²². HERC2 induces the down-regulation of BRCA1 after the irradiation of cells with ultraviolet ray. Thus, the knock-down of HERC2 is likely to generate an opposite phenotype, namely, insensitivity to ultraviolet ray. In order to examine such possibility, the present inventors analyzed whether or not the elimination of HERC2 influences on the survival rate of cells after ultraviolet irradiation.

HeLa cells were transfected with HERC2-specific siRNA, and they were then irradiated with ultraviolet ray. Twenty-four hours after the ultraviolet irradiation, the cell survival rate was measured by the trypan blue exclusion measurement method. The survival rate of the HERC2 knock-down cells after the irradiation with ultraviolet ray of 50 J/m² was approximately 28% of untreated cells at 0 hour after the ultraviolet irradiation. In contrast, the survival rate of control cells was approximately 8.5%. FIG. 5C shows representative data of cells observed by phase-contrast microscopy at 24 hours after the ultraviolet irradiation (50 J/m²). Thus, it became clear that if HERC2 is deleted, cells become resistant to ultraviolet irradiation.

### Example 7

### Detection of Cell Cycle-dependent Expression of BRCA1 and HERC2

In the present example, whether or not the interaction of BRCA1 with HERC2 occurs cell cycle-dependently was analyzed. A thymidine double blocking method was used to synchronize a cell cycle (FIG. 6A), and cells were then recovered every certain amount of time, so that the interaction of BRCA1 with HERC2 was detected by the immunoprecipitation method (FIG. 6B).

Immunoprecipitation was performed using an anti-BRCA1 antibody, and detection was then carried out using an anti-HERC2 antibody (FIG. 6B, uppermost column) and an anti-BRCA1 antibody (FIG. 6B, second column). As a result, HERC2 became a peak at 2 to 4 hours after the recovery of the cells, which was considered to be a G2-M phase, and at the same time, the signal of BRCA1 was attenuated.

In addition, changes in the amounts of HERC2 and BRCA1 in a total cell lysate were analyzed by immunoblotting, without using immunoprecipitation. As a result, a change in the protein amount of HERC2 was hardly observed, but a reduction in BRCA1 was confirmed. This result suggests the interaction of HERC2 with BRCA1 that depends on a cell cycle and the subsequent decomposition of BRCA1 due to HERC2.

### Example 8

Change in Interaction of HERC2 with BRCA1 Due to UV Irradiation

In the present example, whether or not the interaction of BRCA1 with HERC2 is changed due to UV irradiation was analyzed (FIG. 7).

In accordance with the procedures of the aforementioned UV irradiation experiment, after UV irradiation for a certain period of time, immunoprecipitation was carried out using an anti-BRCA1 antibody. Thereafter, the interaction of BRCA1 with HERC2 was detected using an anti-HERC2 antibody (FIG. 7, upper panel), and also, BRCA1 was detected using an anti-BRCA1 antibody as a control (FIG. 7, lower panel).

As a result, in the cells irradiated with UV, an increase in the BRCA1 protein and a decrease in the interaction of BRCA1 with HERC2 were confirmed.

### Consideration

BRCA1 acts as a hub protein in a tumor progression pathway, and as a result of the mutagenesis of the germ line of this key gene, the lifetime risk of breast cancer becomes approximately 80% ²³. Accordingly, it was suggested that the down-regulation of the BRCA1 protein by another mechanism is likely to induce sporadic breast cancer^{24, 25}.

The results described in the present specification demonstrate that HERC2 may be associated with a mechanism whereby, in response to DNA damage, HERC2 regulates the cellular localization and stability of BRCA1 and it thereby down-regulates the BRCA1 protein. A biological importance of this mechanism may be that HERC2 is able to induce the decomposition of BRCA1 in a mechanism associated with extranuclear transport. There is a report that the extranuclear transport of BRCA1 and BARD1 to a cytoplasm causes such decomposition. This report supports this concept²⁶. The data of the present inventors show that if BRCA1 is stabilized by HERC2 knock-down, there are generated cells resistant to DNA damage caused by ultraviolet ray. This suggests that the balance between HERC2 and BRCA1 influences on the survival rate of the cells.

There is data showing that cells can be escaped from DNA damage inducible cell death by the knock-down of HERC2. Based on the data, HERC2 is considered to be a tumor-suppressing factor. However, since HERC2 inhibits BRCA1 as a tumor-suppressing factor, it is more reasonable to consider HERC2 as a cancer gene. Otherwise, it is considered that BRCA1 and HERC2 cooperate with each other as housekeeping tumor-suppressing factors.

As a result of the analysis of ethylnitrosourea (ENU) mutagenesis, it was suggested that HERC2 is the most mutable mouse gene locus so far. This suggestion is worthy of attention²⁷. Although a HERC2 huge duplicon is considered to be a pseudogene having a high mutation rate, it is transcribed. Several rjs mutants comprise a HERC2 protein having an intact HECT domain, but they delete 53 amino acids (3716-3768) ranging from positions 3716 to 3768 with respect to the amino acid sequence of a wild-type HERC2 protein.

### REFERENCES

1. Lehman, A. L. et al. A very large protein with diverse functional motifs is deficient in rjs (runty, jerky, sterile) mice. Proc Natl Acad Sci U S A 95, 9436-41 (1998).

2. Ji, Y. et al. The ancestral gene for transcribed, low-copy repeats in the Prader-Willi/Angelman region encodes a large protein implicated in protein trafficking, which is deficient in mice with neuromuscular and spermiogenic abnormalities. Hum Mol Genet 8, 533-42 (1999).

3. Ji, Y. et al. Structure of the highly conserved HERC2 gene and of multiple partially duplicated paralogs in human. Genome Res 10, 319-29 (2000).

4. Chai, J. H. et al. Identification of four highly conserved genes between breakpoint hotspots BP1 and BP2 of the Prader-Willi/Angelman syndromes deletion region that have undergone evolutionary transposition mediated by flanking duplicons. Am J Hum Genet 73, 898-925 (2003).

5. Garcia-Gonzalo, F. R. & Rosa, J. L. The HERC proteins: functional and evolutionary insights. Cell Mol Life Sci 62, 1826-38 (2005).

6. Nicholls, R. D. & Knepper, J. L. Genome organization, function, and imprinting in Prader-Willi and Angelman syndromes. Annu Rev Genomics Hum Genet 2, 153-75 (2001).

7. Lyon, M. F. et al. Genetic and molecular analysis of recessive alleles at the pink-eyed dilution (p) locus of the mouse. Proc Natl Acad Sci U S A 89, 6968-72 (1992).

8. Rinchik, E. M., Carpenter, D. A. & Handel, M. A. Pleiotropy in microdeletion syndromes: neurologic and spermatogenic abnormalities in mice homozygous for the p6H deletion are likely due to dysfunction of a single gene. Proc Natl Acad Sci U S A 92, 6394-8 (1995).

9. Deng, C. X. Roles of BRCA1 in centrosome duplication. Oncogene 21, 6222-7 (2002).

10. Venkitaraman, A. R. Cancer susceptibility and the functions of BRCA1 and BRCA2. Cell 108, 171-82 (2002).

11. Zheng, L., Li, S., Boyer, T. G. & Lee, W. H. Lessons learned from BRCA1 and BRCA2. Oncogene 19, 6159-75 (2000).

12. Cortez, D., Wang, Y., Qin, J. & Elledge, S. J. Requirement of ATM-dependent phosphorylation of brcal in the DNA damage response to double-strand breaks. Science 286, 1162-6 (1999).

13. Tibbetts, R. S. et al. Functional interactions between BRCA1 and the checkpoint kinase ATR during genotoxic stress. Genes Dev 14, 2989-3002 (2000).

14. Scully, R. et al. Dynamic changes of BRCA1 subnuclear location and phosphorylation state are initiated by DNA damage. Cell 90, 425-35 (1997).

15. Zhong, Q. et al. Association of BRCA1 with the hRad50-hMre11-p95 complex and the DNA damage response. Science 285, 747-50 (1999).

16. Arizti, P. et al. Tumor suppressor p53 is required to modulate BRCA1 expression. Mol Cell Biol 20, 7450-9 (2000).

17. MacLachlan, T. K., Dash, B. C., Dicker, D. T. & El-Deiry, W. S. Repression of BRCA1 through a feedback loop involving p53. J Biol Chem 275, 31869-75 (2000).

18. Blagosklonny, M. V. et al. Regulation of BRCA1 by protein degradation. Oncogene 18, 6460-8 (1999).

19. Nishikawa, H. et al. Mass spectrometric and mutational analyses reveal Lys-6-linked polyubiquitin chains catalyzed by BRCA1-BARD1 ubiquitin ligase. J Biol Chem 279, 3916-24 (2004).

20. Abbott, D. W. et al. BRCA1 expression restores radiation resistance in BRCA1-defective cancer cells through enhancement of transcription-coupled DNA repair. J Biol Chem 274, 18808-12 (1999).

21. Ruffner, H., Joazeiro, C. A., Hemmati, D., Hunter, T. & Verma, I. M. Cancer-predisposing mutations within the RING domain of BRCA1: loss of ubiquitin protein ligase activity and protection from radiation hypersensitivity. Proc Natl Acad Sci U S A 98, 5134-9 (2001).

22. Shen, S. X. et al. A targeted disruption of the murine Brca1 gene causes gamma-irradiation hypersensitivity and genetic instability. Oncogene 17, 3115-24 (1998).

23. King, M. C., Marks, J. H. & Mandell, J. B. Breast and ovarian cancer risks due to inherited mutations in BRCA1 and BRCA2. Science 302, 643-6 (2003).

24. Baldassarre, G. et al. Negative regulation of BRCA1 gene expression by HMGA1 proteins accounts for the reduced BRCA1 protein levels in sporadic breast carcinoma. Mol Cell Biol 23, 2225-38 (2003).

25. Catteau, A. & Morris, J. R. BRCA1 methylation: a significant role in tumour development? Semin Cancer Biol 12, 359-371 (2002).

26. Rodriguez, J. A., Schuchner, S., Au, W. W., Fabbro, M. & Henderson, B. R. Nuclear-cytoplasmic shuttling of BARD1 contributes to its proapoptotic activity and is regulated by dimerization with BRCA1. Oncogene 23, 1809-20 (2004).

27. Walkowicz, M. et al. Molecular characterization of radiation- and chemically induced mutations associated with neuromuscular tremors, runting, juvenile lethality, and sperm defects in jdf2 mice. Mamm Genome 10, 870-8 (1999).

28. Fasano, O., Birnbaum, D., Edlund, L., Fogh, J. & Wigler, M. New human transforming genes detected by a tumorigenicity assay. Mol Cell Biol 4, 1695-705 (1984).

29. Hashizume, R. et al. The RING heterodimer BRCA1-BARD1 is a ubiquitin ligase inactivated by a breast cancer-derived mutation. J Biol Chem 276, 14537-40 (2001).

30. Sato, K. et al. Nucleophosmin/B23 is a candidate substrate for the BRCA1-BARD1 ubiquitin ligase. J Biol Chem 279, 30919-22 (2004).

### Sequence Listing Free Text

### SEQ ID NO: 4 Synthetic DNA

SEQ ID NO: 5 Synthetic DNA

1. A method for suppressing a tumor, which is characterized in that it comprises suppressing the expression of HERC2 in a cell.

2. A method for suppressing a tumor, which is characterized in that it comprises inhibiting the interaction between HERC2 and BRCA1 in a cell.

3. The method according to claim 1, wherein the expression of HERC2 is suppressed by siRNA that acts on a HERC2 gene.

4. The method according to claim 2, wherein the interaction between HERC2 and BRCA1 is inhibited by at least one selected from the group consisting of siRNA acting on the HERC2 gene, an antagonist against the HERC2, and an antibody reacting with the HERC2.

5. A method for screening an antitumor agent, which is characterized in that it comprises measuring the expression level of HERC2 in a cell in the presence of a candidate substance and then selecting a substance having antitumor activity using the obtained measurement result as an indicator.

6. A method for screening an antitumor agent, which is characterized in that it comprises measuring the interaction between HERC2 and BRCA1 in the presence of a candidate substance and then selecting a substance having antitumor activity using the obtained measurement result as an indicator.

7. An antitumor agent comprising a substance that suppresses the expression of HERC2 in a cell.

8. The antitumor agent according to claim 7, wherein the substance that suppresses the expression of HERC2 is siRNA that acts on a HERC2 gene.

9. An antitumor agent comprising a substance that inhibits the interaction between HERC2 and BRCA1 in a cell.

10. The antitumor agent according to claim 9, wherein the substance that inhibits the interaction between HERC2 and BRCA1 is at least one selected from the group consisting of siRNA acting on the HERC gene, an antagonist against the HERC2, and an antibody reacting with the HERC2.

11. A method for treating a tumor, which is characterized in that it comprises suppressing the expression of HERC2 in vivo.

12. A method for treating a tumor, which is characterized in that it comprises inhibiting the interaction between HERC2 and BRCA1 in vivo.

13. Use of a substance suppressing the expression of HERC2 in a cell for the production of a pharmaceutical for treating a tumor.

14. Use of a substance inhibiting the interaction between HERC2 and BRCA1 in a cell for the production of a pharmaceutical for treating a tumor.


SEQUENCE LISTING (15 sequences)

SEQ ID NO: 1 - DNA, 15304 bp, Homo sapiens
  CDS (62)..(14566)
gaggcgaggcggccgggccctgcgcgtcaggcctgagacctgggaggaagctggagaaaagatgccctctgaatctttctgtttggctgcccaggctcgcctcgactccaaatggttgaaaacagatatacagcttgcattcacaagagatgggctctgtggtctgtggaatgaaatggttaaagatggagaaattgtatacactggaacagaatcaacccagaacggagagctccctcctagaaaagatgatagtgtcgaaccaagtggaacaaagaaagaagatctgaatgacaaagagaaaaaagatgaagaagaaactcctgcacctatatatagggccaagtcaattctggacagctgggtatggggcaagcaaccagatgtgaatgaactgaaggagtgtctttctgtgctggttaaagagcagcaggccctggccgtccagtcagccaccaccaccctctcagccctgcgactcaagcagaggctggtgatcttggagcgctatttcattgccttgaatagaaccgtttttcaggagaatgtcaaagttaagtggaaaagcagcggtatttctctgcctcctgtggacaaaaaaagttcccggcctgcgggcaaaggtgtggaggggctcgccagagtgggatcccgagcggcgctgtcttttgcctttgccttcctgcgcagggcctggcgatcaggcgaggatgcggacctctgcagtgagctgttgcaggagtccctggacgccctgcgagcacttcccgaggcctcgctctttgacgagagcaccgtgtcctctgtgtggctggaggtggtggagagagcgaccaggttcctcaggtccgtcgtgacgggggatgttcacggaacgccagccaccaaagggccaggaagcatccccctgcaggaccagcacttggccctggccatcctgctggagctggctgtgcagagaggcacgctgagccaaatgttgtctgccatcctgttgttgcttcagctgtgggacagcggggcacaggagactgacaatgagcgttccgcccagggcaccagcgccccacttttgcccttgctgcaaaggttccagagcatcatttgcaggaaggatgcaccccactccgagggcgacatgcaccttttgtctggccctctgagccccaatgagagtttcctgaggtacctcacccttccacaagacaacgagcttgccattgatctgcgacaaacggcggttgttgtcatggcccatttagaccgtctggctacgccctgtatgcctccgctgtgtagctctccgacatctcataagggatcattgcaagaggtcataggttgggggttaataggatggaaatactatgccaatgtgattggtccaatccagtgcgaaggcctggccaacctgggagtcacacagattgcctgtgcagagaagcgtttcctgattctgtcacgcaatggccgcgtgtacacacaggcctataatagtgacacgctggccccacagctggtccaaggccttgcctccagaaacattgtaaaaattgctgcccattctgatggtcaccactacctagccttggctgctactggagaggtgtactcctggggctgtggggacggcggacggctgggccatggggacactgtgcctttggaggagcctaaggtgatctccgccttctctggaaagcaggccgggaagcacgtggtgcacatcgcttgcgggagcacttacagtgcggccatcactgccgagggggagctgtacacctggggccgcgggaactacggccggctgggccatggctccagtgaggacgaggccattccgatgctggtagccgggcttaaaggactgaaggtcatcgatgtggcgtgtgggagtggggatgctcaaaccctggctgtcactgagaacgggcaagtgtggtcttggggagatggtgactatgggaaattgggcagaggtggtagtgatggctgcaaaaccccaaagctgattgaaaagcttcaagacttggatgtggtcaaagtccgctgtggaagtcagttttccattgctttgacgaaagatggccaagtttattcatggggaaaaggtgacaaccagagacttggacatggaacagaggaacatgttcgttatccaaaactcttagaaggcttgcaagggaagaaggtgattgatgtggctgcaggctccacccactgcctggctctgactgaggacagcgaggtccacagctgggggagcaacgaccagtgccagcactttgacaccttgcgcgtgaccaagccagaacctgcagcattgccaggactggacaccaaacacatagtgggaattgcctgtgggcctgcccagagctttgcttggtcatcatgttctgagtggtccattggcctccgtgtcccttttgtggtggacatctgctcaatgacttttgagcagctagatctcctgcttcggcaggtgagtgaggggatggatggctccgcggactggcccccgccccaggagaaagagtgtgtggccgtggcaacgctgaatcttctacgacttcagttgcatgctgccattagtcaccaggttgacccggaattccttggtttaggtctgggcagcatcctcctgaacagcctgaagcagacggtggtgaccctggccagcagtgcgggcgtgctgagcaccgtgcagtcggccgcccaggccgtgctgcagagtggctggtccgtgctgctgcccaccgcggaggagcgggcccgggcactctctgctctcctgccctgcgcagtttcaggcaatgaagtgaacataagtccaggtcgtcgattcatgattgatcttctggtgggcagcttgatggctgatggagggttggagtcagccttacacgcagccattactgcagagatccaggatattgaagccaaaaaagaagcacagaaggaaaaagaaattgatgaacaggaagcgaatgcctcaacatttcatagaagcaggactccactggataaagaccttattaatacggggatctgtgagtcttctggcaaacagtgtttgcctctggttcagctcatacaacagcttcttagaaacatcgcttctcagactgtagccagattgaaagatgttgcccgtcggatttcatcatgtctggactttgagcaacacagtcgtgaaagatctgcttcattggattggttactgcgtttccaacgtttgcttattagtaaactttatccaggagaaagtattggtcagacctcagatatttctagtccagagctaatgggtgttggttccttgctgaagaagtacacagccctcctgtgcacgcacattggagatatactgcctgtggccgccagcattgcttctaccagctggcggcacttcgcggaggtggcttacattgtggaaggggactttactggtgttctccttccagaactagtagtttctatagtgcttctgctcagtaaaaatgctgatctcatgcaagaggctggagctgtacctctgctgggtggcctgttggaacatctggatcggttcaaccatctggcaccaggaaaggaacgggatgatcatgaagagttagcctggcctggcataatggagtcattttttacaggtcagaactgtagaaataatgaggaagtgacacttatacgcaaagctgatttggagaaccataataaagatggaggcttctggactgtgattgacgggaaggtgtatgatataaaggacttccagacacagtcgttaacaggaaatagtattcttgctcagtttgcaggggaagacccagtggtagctttggaagctgctttgcagtttgaagacacccgggaatccatgcacgcgttttgtgttggccagtatttggagcctgaccaagaaatcgtcaccataccagatctggggagtctctcttcacctctgatagacacagagaggaatctgggcctgcttctcggattacacgcttcgtatttggcaatgagcacaccgctgtctcctgtcgagattgaatgtgccaaatggcttcagtcatccatcttctctggaggcctgcagaccagccagatccactacaggtacaacgaggagaaagacgaggaccactgcagctccccagggggcacacctgccagcaaatctcgactctgctcccacagacgggccctgggggaccattcccaggcatttctgcaagccattgcagacaacaacattcaggatcacaacgtgaaggactttttgtgtcaaatagaaaggtactgtaggcagtgccatttgaccacaccgatcatgtttccccccgagcatcccgtggaagaggtcggtcgcttgttgttatgttgcctcttaaaacatgaagatttaggtcatgtggcattatctttagttcatgcaggtgcacttggtattgagcaagtaaagcacagaacgttgcctaagtcagtggtggatgtttgtagagttgtctaccaagcaaaatgttcgctcattaagactcatcaagaacagggccgttcttacaaggaggtctgcgctcctgtcatcgaacgtttgagattcctctttaatgaattgagacctgctgtttgtaatgacctctctataatgtctaagtttaaattgttaagttctttgccccgttggaggaggatagctcaaaagataattcgagaacgaaggaaaaagagagttcctaagaagccagaatctatggatgatgaagaaaaaattggaaacgaagagagtgatttagaagaagcttgcattttgcctcatagtccaataaatgtggacaagagacccattgcaattaaatcacccaaggacaaatggcagccgctgttgagtactgttacaggtgttcacaaatacaagtggttgaagcagaatgtgcagggtctttatccgcagtctccactcctcagtacaattgctgaatttgcccttaaagaagagccagtggatgtggaaaaaatgagaaagtgcctactaaaacagttggagagagcagaggttcgcctggaagggatagatacaattttaaaactggcgagcaagaatttcttacttccatctgtgcagtatgcgatgttttgtggatggcaaagacttattcctgagggaatcgatataggggaacctcttactgattgtttaaaggatgttgatttgatcccgccttttaatcggatgctgctggaagtcacctttggcaagctgtacgcttgggctgtacagaacattcgaaatgttttgatggatgccagtgccacatttaaagagcttggtatccagccggttcccctccaaaccatcaccaatgagaacccgtcaggaccgagcctggggaccatcccgcaagcccgcttcctcctggtgatgctcagcatgctcaccctgcagcacggcgcaaacaacctcgaccttctgctcaattccggcatgctggccctcacgcagacggcactgcgcctgattggccccagttgtgacaacgttgaggaagatatgaatgcttctgctcaaggtgcttctgccacagttttggaagaaacaaggaaggaaacggctcctgtgcagctccctgtttcaggaccagaactggctgccatgatgaagattggaacaagggtcatgagaggtgtggactggaaatggggcgatcaggatgggcctcctccaggcctaggccgcgtgattggtgagctgggagaggacggatggataagagtccagtgggacacaggcagcaccaactcctacaggatggggaaagaaggaaaatacgacctcaagctggcagagctgccggctgctgcacagccctcagcagaggattcggacacagaggatgactctgaagccgaacaaactgaaaggaacattcaccccactgcaatgatgtttaccagcactattaacttactgcagactctttgtctgtctgctggagttcatgctgagatcatgcagagcgaagccaccaagactttatgcggactgctgcgaatgttagtggaaagcggaacgacggacaagacatcttctccaaacaggctggtgtacagggagcaacaccggagctggtgcacgctggggtttgtgcggagcatcgctctcacgccgcaggtatgcggcgccctcagctccccgcagtggatcacgctgctcatgaaggtcgtggaagggcacgcacccttcactgccacctcgctgcagaggcagatcttagctgtgcatttgttgcaagcagtccttccatcatgggacaagaccgaaagggcgagggacatgaaatgcctcgtggagaagctgtttgacttcttgggaagcttgctcactacctgctcctctgacgtgccattactcagagagtccacgctgaggcggcgcagggtgcgcccgcaggcctcgctgactgccacccacagcagcacactggcggaggaggtggtggcactgctgcgcacgctgcactccctgactcagtggaatgggctcatcaacaagtacatcaactcccagctccgctccatcacccacagctttgtgggaaggccttccgaaggggcccagttagaggactacttccccgactccgagaaccctgaagtggggggcctcatggcagtcctggctgtgattggaggcatcgatggtcgcctgcgcctgggcggtcaagttatgcacgatgagtttggagaaggcactgtgactcgcatcaccccaaagggcaaaatcaccgtgcagttctctgacatgcggacgtgtcgcgtttgcccattgaatcagctgaaaccactccctgccgtggcctttaatgtgaacaacctgcccttcacagagcccatgctgtctgtctgggctcagttggtgaacctcgctggaagcaagttagaaaagcacaaaataaagaaatcgactaaacaggcctttgcaggacaagtggacctggacctgctgcggtgccagcagttgaagctatacatcctgaaagcaggtcgggcgctgctctcccaccaggataaactgcggcagatcctgtctcagccagctgttcaggagactggaactgttcacacagatgatggagcagtggtatcacctgaccttggggacatgtctcctgaagggccgcagccccccatgatcctcttgcagcagctgctggcctcggccacccagccgtctcctgtgaaggccatatttgataaacaggaacttgaggctgctgcactggccgtttgccagtgcttggctgtggagtccactcacccttcgagcccaggatttgaagactgcagctccagtgaggccaccacgcctgtcgccgtgcagcacatccaccctgccagagtgaagaggcgcaagcagtcgcccgttcccgctctgccgatcgtggtgcagctcatggagatgggattttccagaaggaacatcgagtttgccctgaagtctctcactggtgcttccgggaatgcatccagcttgcctggtgtggaagccttggtcgggtggctgctggaccactccgacatacaggtcacggagctctcagatgcagacacggtgtccgacgagtattctgacgaggaggtggtggaggacgtggatgatgccgcctactccatgtctactggtgctgttgtgacggagagccagacgtacaaaaaacgagctgatttcttgagtaatgatgattatgctgtatatgtgagagagaatattcaggtgggaatgatggttagatgctgccgagcgtatgaagaagtgtgcgaaggtgatgttggcaaagtcatcaagctggacagagatggattgcatgatctcaatgtgcagtgtgactggcagcagaaagggggcacctactgggttaggtacattcatgtggaacttataggctatcctccaccaagttcttcttctcacatcaagattggtgataaagtgcgggtcaaagcctctgtcaccacaccaaaatacaaatggggatctgtgactcatcagagtgtgggggttgtgaaagctttcagtgccaatggaaaagatatcattgtcgactttccccagcagtctcactggactgggttgctatcagaaatggagttggtacccagtattcatcctggggttacgtgtgatggatgtcagatgtttcctatcaatggatccagattcaaatgcagaaactgtgatgactttgatttttgtgaaacgtgtttcaagaccaaaaaacacaataccaggcatacatttggcagaataaatgaaccaggtcagtctgcggtattttgtggccgttctggaaaacagctgaagcgttgccacagcagccagccaggcatgctgctggacagctggtcccgcatggtgaagagcctgaatgtgtcgtcctccgtgaaccaggcatcccgtctcattgacggcagcgagccctgctggcagtcatcggggtcgcaaggaaagcactggattcgtttggagattttcccagatgttcttgttcatagattaaaaatgatcgtagatcctgctgacagtagctacatgccgtccctggttgtagtgtcaggtggaaattccctgaataaccttattgaactaaagacaatcaatattaacccttctgacaccacagtgccccttctgaatgactacacagagtatcacaggtatattgaaattgctataaagcagtgcaggagctcaggaatcgattgtaaaatccatggtctcatcctgctgggacggatccgtgcagaagaggaagatttggctgcagttcctttcttagcttcggataatgaagaggaggaggatgagaaaggcaacagcggaagcctcattagaaagaaggctgctgggctggaatcagcagctacgataagaaccaaggtgtttgtgtggggcctgaatgacaaggaccagctgggcgggctgaaaggctccaagataaaggttccttcgttctctgagacactgtcagctttgaatgtggtacaggtggctggtggatctaaaagtttgtttgcagtgactgtggaagggaaggtgtatgcctgtggagaagccacgaatggccggctggggctgggcatttccagcgggacggtgcccatcccacggcagatcacagctctcagcagctacgtggtcaagaaggtggctgttcactcaggtggccggcacgcgacggctttaactgtcgatggaaaagtgttttcgtggggcgaaggtgacgatggaaaacttggacacttcagcagaatgaactgtgacaaaccaaggctgatcgaggccctgaaaaccaagcgtatccgggatatcgcctgtgggagctcgcacagcgcagccctcacatccagcggagaactgtacacctggggcctcggcgagtacggccggctgggacatggggataatacgacacagctaaagcccaaaatggtgaaagtccttctcggtcacagagtaatccaggttgcatgtgggagtagagacgcgcagaccctggctctgaccgatgaaggtttggtattttcctggggtgatggtgactttggaaaactgggccggggcggaagtgaaggctgtaacattccccagaacattgagagactaaatggacagggggtgtgccagattgagtgtggagctcagttctccctggcgctcaccaagtctggagtggtgtggacatggggaaagggggattacttcagattgggccacggctctgacgtgcacgtgcggaaaccacaggtggtggaagggctgagagggaagaagatcgtgcatgtggctgtcggggccctgcactgcctggcggtcacggactcggggcaggtgtatgcttggggtgacaacgaccacggccagcagggcaatggcacgaccacggttaacaggaagcccacactcgtgcaaggcttagaaggccagaagatcacacgcgtggcttgtgggtcgtcccacagtgtggcgtggacaactgtggatgtggccacgccctctgtccacgagcccgtcctcttccagactgcaagagacccgttaggtgcttcctacttaggcgtgccctcagatgctgattcttctgctgccagtaataaaataagtggtgcaagtaattctaagccaaatcgcccttctcttgccaagattctcttgtcattggatggaaatctggccaaacagcaggccttatcgcatattcttacagcattgcaaatcatgtatgccagagatgctgttgtcggggccctgatgccggccgccatgatcgccccggtggagtgcccctcgttctcctcggcggccccttccgacgcatctgcgatggctagtcccatgaatggagaagaatgcatgctggctgttgatatcgaagacagactgagtccaaatccatggcaagaaaagagagagattgtttcctctgaggacgcagtgaccccctctgcagtgactccgtcggccccctcagcctccgctcggccttttatcccagtgacggatgacctgggagctgcaagcatcattgcagaaaccatgaccaaaaccaaagaggatgttgaaagccaaaataaagcagcaggtccggagcctcaggccttggatgagttcaccagtctgctgattgcggatgacactcgtgtggtggtagacctgctcaagctgtcagtgtgcagccgggccggggacaggggcagggatgtgctctccgcggtgctttccggcatggggaccgcctacccacaggtggcagatatgctgttggagctctgtgtcaccgagttggaggatgtggccacagactcgcagagcggccgcctctcttctcagcctgtggtggtggagagtagccacccttacaccgacgacacctccaccagtggcacagtgaagataccaggtgcagaaggactcagggtagaatttgaccggcagtgctccacagagaggcgccacgaccctctcacagtcatggacggcgtcaacaggatcgtctccgtgcggtcaggccgagagtggtccgactggtccagcgagctgcgcatcccaggggatgagttaaagtggaagttcatcagcgatgggtctgtgaatggctggggctggcgcttcaccgtctatcccatcatgccagctgctggccctaaagaactcctctctgaccgctgcgtcctctcctgtccatccatggacttggtgacgtgtctgttagacttccgactcaaccttgcctctaacagaagcatcgtccctcgccttgcggcctcgctggcagcttgtgcacagctgagtgccctagctgccagtcacagaatgtgggcccttcagagactgaggaagctgcttacaactgaatttgggcagtcaattaacataaataggctgcttggagaaaatgatggggaaacaagagctttgagttttacaggtagtgctcttgctgctttggtgaaaggtcttccagaagctttgcaaaggcagtttgaatatgaagatcctattgtgaggggtggcaaacagctgctccacagcccattctttaaggtactagtagctcttgcttgtgacctggagctggacactctgccttgctgtgccgagacgcacaagtgggcctggttccggaggtactgcatggcctcccgtgttgctgtggcccttgacaaaagaacaccgttgccccgtctgtttcttgatgaggtggctaagaaaattcgtgaattaatggcagacagcgaaaacatggatgttctgcatgagagccatgacatttttaaaagagagcaagacgaacaacttgtgcagtggatgaacaggcgaccagatgactggactctctctgctggtggcagtggaacaatttatggatggggacataatcacaggggccagctcgggggcattgaaggcgcaaaagtcaaagttcccactccctgtgaagcccttgcaactctcagacccgtgcagttaatcggaggggaacagaccctctttgctgtgacggctgatgggaagctgtatgccactgggtatggtgcaggtggcagactaggcattggagggacagagtcggtgtccaccccaacattgcttgaatccattcagcatgtgtttattaagaaagtagctgtgaactctggaggaaagcactgccttgccctgtcttcagaaggagaagtttactcttggggtgaggcagaagatgggaagttggggcatggcaacagaagtccgtgtgaccgccctcgtgtcatcgagtctctgagaggaattgaagtggtcgatgttgctgctggcggagcccacagcgcctgtgtcacagcagccggggacctctacacatggggcaaaggccgctacggccggctggggcacagcgacagtgaggaccagctgaagccgaagctggtggaggcgctgcagggccaccgtgtggttgacatcgcctgtggcagtggagatgcccagaccctctgcctcacagatgacgacactgtctggtcctggggggacggggactacggcaagctcggccggggaggcagcgatggctgtaaagtgcctatgaagattgattctcttactggtcttggagtagttaaagtggaatgcggatcccagttttctgttgcccttaccaaatctggagctgtttatacctggggcaaaggcgattatcacaggttgggccatggatcagatgaccatgttcgaaggcctcggcaggtccaagggttgcaggggaagaaagtcatcgccatcgccactggctccctgcactgtgtgtgctgcacagaggatggtgaggtttatacatggggcgacaatgatgagggacaactgggagacggaaccaccaatgccatccagaggcctcggttggtagctgcccttcagggtaagaaggtcaaccgtgtggcctgtggctcagcacataccctcgcctggtcgaccagcaagcccgccagtgctggcaaactccctgcacaggtccccatggagtacaatcacctgcaggagatccccatcattgcgctgaggaaccgtctgctgctgctgcaccacctctccgagctcttctgcccctgcatccccatgttcgacctggaaggctcgctcgacgaaactggactcgggccttctgttgggttcgacactctccgaggaattctgatatcccagggaaaggaggcggctttccggaaagtagtacaagcaactatggtacgcgatcgtcagcatggccccgtcgtggagctgaaccgcatccaggtcaaacgatcaaggagcaaaggcgggctggccggccccgacggcaccaagtctgtctttgggcagatgtgtgctaagatgagctcgtttggtcccgacagcctcctccttcctcaccgtgtctggaaagtcaagtttgtgggtgaatctgtggatgactgtgggggcggctacagcgagtccatagctgagatctgtgaggagctgcagaacggactcacgcccctgctgatcgtgacacccaacgggagggatgagtctggggccaaccgagactgctacctgctcagcccggccgccagagcacccgtgcacagcagcatgttccgcttcctgggtgtgttgctgggcattgccatccgaaccgggagtcccctgagcctcaaccttgccgagcctgtctggaagcagctggctgggatgagcctcaccatcgcggacctcagtgaggttgataaggattttattcctggactcatgtacatccgagacaatgaagccacctcagaggagtttgaagccatgagcctgcccttcacagtgccaagtgccagtggccaggacattcagttgagctccaagcacacacacatcaccctggacaaccgcgcggagtacgtgcggctggcgataaactatagactccatgaatttgatgagcaggtggctgctgttcgggaaggaatggcccgcgttgtgcctgttcccctcctctctctgttcaccggctacgaactggagacgatggtgtgtggcagccctgacatcccgctgcaccttctcaagtcggtggccacctataaaggcatcgagccttccgcatcgctgatccagtggttctgggaggtgatggagtccttctccaacacagagcgctctcttttccttcgcttcgtctggggccggacgaggctgcccaggaccatcgccgacttccggggccgagacttcgtcatccaggtgttggataaatacaaccctccagaccacttcctccctgagtcctacacctgtttcttcttgctgaagctgcccaggtattcctgcaagcaggtgctggaggagaagctcaagtacgccatccacttctgcaagtccatagacacagatgactacgctcgcatcgcacttacaggagagccagccgccgacgacagcagcgacgattcagataacgaggatgtcgactcctttgcttcggactctacacaagattatttaacaggacactaagatggggaaacgtcctcgtgagatgagagcctgagccaggcagcagagcactcgctgctgtgtagactgtaggctgcctggtgtgtctgatgagaagcgtccgtcctcgagccaggcgggaggagggagtggagagactgactggccgtgatgggaatgacagtgagaaggtccgcctgtgcgcgtggaacactgtggacgctcgacttccaagggtcttctcacccgtaatgctgcattacatgtaggactgtgtttactaaagtgtgtaaatgtttatataaataccaaattgcagcatccccaaaatgaataaagcctttttacttgtgggtgcaatcgatttttttttctttctcctttctttcaagtgtcgtgagtcgtcttgattgtatattggaaataactgtgtaacaaatcgtattataaatatttcaattaattttactctgaatttgtttattaaaagacttttgaacatgaaatgattagtattacttgaatgcatccagaggatatttaaaccaaaatgaaaaaccagaaggccatttggtgtcccctctcccaggtgtccccttgtagcatatgcattatgtcatctgaattgaggcctttctgtgaacagcatcataacttctatcatggaaagtgtactatatataatgtttgtgtcatgtatatgcctaaattttaatcatctataaataaaacatctgacataaaaaaaaaaaaaaaaaa

SEQ ID NO: 2 - protein, 4834 aa, Homo sapiens
MPSESFCLAAQARLDSKWLKTDIQLAFTRDGLCGLWNEMVKDGEIVYTGTESTQNGELPPRKDDSVEPSGTKKEDLNDKEKKDEEETPAPIYRAKSILDSWVWGKQPDVNELKECLSVLVKEQQALAVQSATTTLSALRLKQRLVILERYFIALNRTVFQENVKVKWKSSGISLPPVDKKSSRPAGKGVEGLARVGSRAALSFAFAFLRRAWRSGEDADLCSELLQESLDALRALPEASLFDESTVSSVWLEVVERATRFLRSVVTGDVHGTPATKGPGSIPLQDQHLALAILLELAVQRGTLSQMLSAILLLLQLWDSGAQETDNERSAQGTSAPLLPLLQRFQSIICRKDAPHSEGDMHLLSGPLSPNESFLRYLTLPQDNELAIDLRQTAVVVMAHLDRLATPCMPPLCSSPTSHKGSLQEVIGWGLIGWKYYANVIGPIQCEGLANLGVTQIACAEKRFLILSRNGRVYTQAYNSDTLAPQLVQGLASRNIVKIAAHSDGHHYLALAATGEVYSWGCGDGGRLGHGDTVPLEEPKVISAFSGKQAGKHVVHIACGSTYSAAITAEGELYTWGRGNYGRLGHGSSEDEAIPMLVAGLKGLKVIDVACGSGDAQTLAVTENGQVWSWGDGDYGKLGRGGSDGCKTPKLIEKLQDLDVVKVRCGSQFSIALTKDGQVYSWGKGDNQRLGHGTEEHVRYPKLLEGLQGKKVIDVAAGSTHCLALTEDSEVHSWGSNDQCQHFDTLRVTKPEPAALPGLDTKHIVGIACGPAQSFAWSSCSEWSIGLRVPFVVDICSMTFEQLDLLLRQVSEGMDGSADWPPPQEKECVAVATLNLLRLQLHAAISHQVDPEFLGLGLGSILLNSLKQTVVTLASSAGVLSTVQSAAQAVLQSGWSVLLPTAEERARALSALLPCAVSGNEVNISPGRRFMIDLLVGSLMADGGLESALHAAITAEIQDIEAKKEAQKEKEIDEQEANASTFHRSRTPLDKDLINTGICESSGKQCLPLVQLIQQLLRNIASQTVARLKDVARRISSCLDFEQHSRERSASLDWLLRFQRLLISKLYPGESIGQTSDISSPELMGVGSLLKKYTALLCTHIGDILPVAASIASTSWRHFAEVAYIVEGDFTGVLLPELVVSIVLLLSKNADLMQEAGAVPLLGGLLEHLDRFNHLAPGKERDDHEELAWPGIMESFFTGQNCRNNEEVTLIRKADLENHNKDGGFWTVIDGKVYDIKDFQTQSLTGNSILAQFAGEDPVVALEAALQFEDTRESMHAFCVGQYLEPDQEIVTIPDLGSLSSPLIDTERNLGLLLGLHASYLAMSTPLSPVEIECAKWLQSSIFSGGLQTSQIHYRYNEEKDEDHCSSPGGTPASKSRLCSHRRALGDHSQAFLQAIADNNIQDHNVKDFLCQIERYCRQCHLTTPIMFPPEHPVEEVGRLLLCCLLKHEDLGHVALSLVHAGALGIEQVKHRTLPKSVVDVCRVVYQAKCSLIKTHQEQGRSYKEVCAPVIERLRFLFNELRPAVCNDLSIMSKFKLLSSLPRWRRIAQKIIRERRKKRVPKKPESMDDEEKIGNEESDLEEACILPHSPINVDKRPIAIKSPKDKWQPLLSTVTGVHKYKWLKQNVQGLYPQSPLLSTIAEFALKEEPVDVEKMRKCLLKQLERAEVRLEGIDTILKLASKNFLLPSVQYAMFCGWQRLIPEGIDIGEPLTDCLKDVDLIPPFNRMLLEVTFGKLYAWAVQNIRNVLMDASATFKELGIQPVPLQTITNENPSGPSLGTIPQARFLLVMLSMLTLQHGANNLDLLLNSGMLALTQTALRLIGPSCDNVEEDMNASAQGASATVLEETRKETAPVQLPVSGPELAAMMKIGTRVMRGVDWKWGDQDGPPPGLGRVIGELGEDGWIRVQWDTGSTNSYRMGKEGKYDLKLAELPAAAQPSAEDSDTEDDSEAEQTERNIHPTAMMFTSTINLLQTLCLSAGVHAEIMQSEATKTLCGLLRMLVESGTTDKTSSPNRLVYREQHRSWCTLGFVRSIALTPQVCGALSSPQWITLLMKVVEGHAPFTATSLQRQILAVHLLQAVLPSWDKTERARDMKCLVEKLFDFLGSLLTTCSSDVPLLRESTLRRRRVRPQASLTATHSSTLAEEVVALLRTLHSLTQWNGLINKYINSQLRSITHSFVGRPSEGAQLEDYFPDSENPEVGGLMAVLAVIGGIDGRLRLGGQVMHDEFGEGTVTRITPKGKITVQFSDMRTCRVCPLNQLKPLPAVAFNVNNLPFTEPMLSVWAQLVNLAGSKLEKHKIKKSTKQAFAGQVDLDLLRCQQLKLYILKAGRALLSHQDKLRQILSQPAVQETGTVHTDDGAVVSPDLGDMSPEGPQPPMILLQQLLASATQPSPVKAIFDKQELEAAALAVCQCLAVESTHPSSPGFEDCSSSEATTPVAVQHIHPARVKRRKQSPVPALPIVVQLMEMGFSRRNIEFALKSLTGASGNASSLPGVEALVGWLLDHSDIQVTELSDADTVSDEYSDEEVVEDVDDAAYSMSTGAVVTESQTYKKRADFLSNDDYAVYVRENIQVGMMVRCCRAYEEVCEGDVGKVIKLDRDGLHDLNVQCDWQQKGGTYWVRYIHVELIGYPPPSSSSHIKIGDKVRVKASVTTPKYKWGSVTHQSVGVVKAFSANGKDIIVDFPQQSHWTGLLSEMELVPSIHPGVTCDGCQMFPINGSRFKCRNCDDFDFCETCFKTKKHNTRHTFGRINEPGQSAVFCGRSGKQLKRCHSSQPGMLLDSWSRMVKSLNVSSSVNQASRLIDGSEPCWQSSGSQGKHWIRLEIFPDVLVHRLKMIVDPADSSYMPSLVVVSGGNSLNNLIELKTININPSDTTVPLLNDYTEYHRYIEIAIKQCRSSGIDCKIHGLILLGRIRAEEEDLAAVPFLASDNEEEEDEKGNSGSLIRKKAAGLESAATIRTKVFVWGLNDKDQLGGLKGSKIKVPSFSETLSALNVVQVAGGSKSLFAVTVEGKVYACGEATNGRLGLGISSGTVPIPRQITALSSYVVKKVAVHSGGRHATALTVDGKVFSWGEGDDGKLGHFSRMNCDKPRLIEALKTKRIRDIACGSSHSAALTSSGELYTWGLGEYGRLGHGDNTTQLKPKMVKVLLGHRVIQVACGSRDAQTLALTDEGLVFSWGDGDFGKLGRGGSEGCNIPQNIERLNGQGVCQIECGAQFSLALTKSGVVWTWGKGDYFRLGHGSDVHVRKPQVVEGLRGKKIVHVAVGALHCLAVTDSGQVYAWGDNDHGQQGNGTTTVNRKPTLVQGLEGQKITRVACGSSHSVAWTTVDVATPSVHEPVLFQTARDPLGASYLGVPSDADSSAASNKISGASNSKPNRPSLAKILLSLDGNLAKQQALSHILTALQIMYARDAVVGALMPAAMIAPVECPSFSSAAPSDASAMASPMNGEECMLAVDIEDRLSPNPWQEKREIVSSEDAVTPSAVTPSAPSASARPFIPVTDDLGAASIIAETMTKTKEDVESQNKAAGPEPQALDEFTSLLIADDTRVVVDLLKLSVCSRAGDRGRDVLSAVLSGMGTAYPQVADMLLELCVTELEDVATDSQSGRLSSQPVVVESSHPYTDDTSTSGTVKIPGAEGLRVEFDRQCSTERRHDPLTVMDGVNRIVSVRSGREWSDWSSELRIPGDELKWKFISDGSVNGWGWRFTVYPIMPAAGPKELLSDRCVLSCPSMDLVTCLLDFRLNLASNRSIVPRLAASLAACAQLSALAASHRMWALQRLRKLLTTEFGQSININRLLGENDGETRALSFTGSALAALVKGLPEALQRQFEYEDPIVRGGKQLLHSPFFKVLVALACDLELDTLPCCAETHKWAWFRRYCMASRVAVALDKRTPLPRLFLDEVAKKIRELMADSENMDVLHESHDIFKREQDEQLVQWMNRRPDDWTLSAGGSGTIYGWGHNHRGQLGGIEGAKVKVPTPCEALATLRPVQLIGGEQTLFAVTADGKLYATGYGAGGRLGIGGTESVSTPTLLESIQHVFIKKVAVNSGGKHCLALSSEGEVYSWGEAEDGKLGHGNRSPCDRPRVIESLRGIEVVDVAAGGAHSACVTAAGDLYTWGKGRYGRLGHSDSEDQLKPKLVEALQGHRVVDIACGSGDAQTLCLTDDDTVWSWGDGDYGKLGRGGSDGCKVPMKIDSLTGLGVVKVECGSQFSVALTKSGAVYTWGKGDYHRLGHGSDDHVRRPRQVQGLQGKKVIAIATGSLHCVCCTEDGEVYTWGDNDEGQLGDGTTNAIQRPRLVAALQGKKVNRVACGSAHTLAWSTSKPASAGKLPAQVPMEYNHLQEIPIIALRNRLLLLHHLSELFCPCIPMFDLEGSLDETGLGPSVGFDTLRGILISQGKEAAFRKVVQATMVRDRQHGPVVELNRIQVKRSRSKGGLAGPDGTKSVFGQMCAKMSSFGPDSLLLPHRVWKVKFVGESVDDCGGGYSESIAEICEELQNGLTPLLIVTPNGRDESGANRDCYLLSPAARAPVHSSMFRFLGVLLGIAIRTGSPLSLNLAEPVWKQLAGMSLTIADLSEVDKDFIPGLMYIRDNEATSEEFEAMSLPFTVPSASGQDIQLSSKHTHITLDNRAEYVRLAINYRLHEFDEQVAAVREGMARVVPVPLLSLFTGYELETMVCGSPDIPLHLLKSVATYKGIEPSASLIQWFWEVMESFSNTERSLFLRFVWGRTRLPRTIADFRGRDFVIQVLDKYNPPDHFLPESYTCFFLLKLPRYSCKQVLEEKLKYAIHFCKSIDTDDYARIALTGEPAADDSSDDSDNEDVDSFASDSTQDYLTGH

SEQ ID NO: 3 - protein, 581 aa, Homo sapiens
IATGSLHCVCCTEDGEVYTWGDNDEGQLGDGTTNAIQRPRLVAALQGKKVNRVACGSAHTLAWSTSKPASAGKLPAQVPMEYNHLQEIPIIALRNRLLLLHHLSELFCPCIPMFDLEGSLDETGLGPSVGFDTLRGILISQGKEAAFRKVVQATMVRDRQHGPVVELNRIQVKRSRSKGGLAGPDGTKSVFGQMCAKMSSFGPDSLLLPHRVWKVKFVGESVDDCGGGYSESIAEICEELQNGLTPLLIVTPNGRDESGANRDCYLLSPAARAPVHSSMFRFLGVLLGIAIRTGSPLSLNLAEPVWKQLAGMSLTIADLSEVDKDFIPGLMYIRDNEATSEEFEAMSLPFTVPSASGQDIQLSSKHTHITLDNRAEYVRLAINYRLHEFDEQVAAVREGMARVVPVPLLSLFTGYELETMVCGSPDIPLHLLKSVATYKGIEPSASLIQWFWEVMESFSNTERSLFLRFVWGRTRLPRTIADFRGRDFVIQVLDKYNPPDHFLPESYTCFFLLKLPRYSCKQVLEEKLKYAIHFCKSIDTDDYARIALTGEPAADDSSDDSDNEDVDSFASDSTQDYLTGH

SEQ ID NO: 4 - DNA, 26 bp, Artificial
  synthetic DNA
taggatccccttaccaaatctggagc

SEQ ID NO: 5 - DNA, 26 bp, Artificial
  synthetic DNA
tagctctcatctctcgaggacgtttc

SEQ ID NO: 6 - protein, 24 aa, Homo sapiens
DAPHSEGDMHLLSGPLSPNESFLR

SEQ ID NO: 7 - protein, 35 aa, Homo sapiens
GLKVIDVACGSGDAQTLAVTENGQVWSWGDGDYGK

SEQ ID NO: 8 - protein, 27 aa, Homo sapiens
LIPEGIDIGEPLTDCLKDVDLIPPFNR

SEQ ID NO: 9 - protein, 30 aa, Homo sapiens
LIGPSCDNVEEDMNASAQGASATVLEETRK

SEQ ID NO: 10 - protein, 18 aa, Homo sapiens
QAFAGQVDLDLLRCQQLK

SEQ ID NO: 11 - protein, 15 aa, Homo sapiens
DGLHDLNVQCDWQQK

SEQ ID NO: 12 - protein, 17 aa, Homo sapiens
QQALSHILTALQIMYAR

SEQ ID NO: 13 - protein, 15 aa, Homo sapiens
GDYHRLGHGSDDHVR

SEQ ID NO: 14 - protein, 11 aa, Homo sapiens
RPRQVQGLQGK

SEQ ID NO: 15 - protein, 19 aa, Homo sapiens
DCYLLSPAARAPVHSSMFR